(19) United States
(12) Patent Application Publication
(10) Pub. No.: US 2023/0317266 A1
(43) Pub. Date: Oct. 5, 2023

(54) MEDICAL SERVICE SUPPORT DEVICE, MEDICAL SERVICE SUPPORT METHOD, AND PROGRAM

(71) Applicant: FUJIFILM Corporation, Tokyo (JP)

(72) Inventors: Yosuke OHASHI, Tokyo (JP); Shinichi KOMIYAMA, Tokyo (JP); Chieko IWANAGA, Tokyo (JP); Yuma HORI, Tokyo (JP); Tatsuya KOBAYASHI, Tokyo (JP)

(73) Assignee: FUJIFILM Corporation, Tokyo (JP)

(21) Appl. No.: 18/331,942

(22) Filed: Jun. 9, 2023

Related U.S. Application Data
(63) Continuation of application No. PCT/JP2021/042373, filed on Nov. 18, 2021.

(30) Foreign Application Priority Data
Dec. 14, 2020 (JP) 2020-206812

(51) Int. Cl. G16H 40/40; A61B 1/00; A61B 90/70; G16H 40/20

(52) U.S. Cl. CPC G16H 40/40; A61B 1/00062; A61B 90/70; G16H 40/20; A61B 2090/701

(57) ABSTRACT

The medical service support device includes a processor and a memory connected to or built into the processor. The processor is configured to derive, based on standard operation time, during which a medical device that is used in a medical device handling service is being operated, and on standby time for a medical device procedure service included in the medical device handling service, an operation rate reference value of the medical device, and output medical service support information obtained in accordance with a difference degree between an operation rate of the medical device and the operation rate reference value.

### CROSS-REFERENCE TO RELATED APPLICATIONS

This application is a continuation application of International Application No. PCT/JP2021/042373, filed Nov. 18, 2021, the disclosure of which is incorporated herein by reference in its entirety. Further, this application claims priority under 35 USC 119 from Japanese Patent Application No. 2020-206812 filed Dec. 14, 2020, the disclosure of which is incorporated by reference herein.

### BACKGROUND

### 1. Technical Field

The present invention relates to a medical service support device, a medical service support method, and a program.

### 2. Related Art

JP2010-039560A discloses a medical device management support device including: an examination schedule storing unit that stores an examination schedule including a start time point and an end time point of each examination; a washing capability storing unit that stores washing capability information that defines washing capability of a washing device that washes an scope used in the examination; a washing schedule generation unit that generates a washing schedule for washing a used scope, which is generated at the end of each examination, with the washing device having washing capability defined based on the washing capability information stored in the washing capability storing unit according to the examination schedule stored in the examination schedule storing unit; and an scope determination unit that determines whether or not there is a shortage of scopes to be used in each examination according to an examination start time point specified through the examination schedule and a washing end time point specified through the washing schedule.

### SUMMARY

One embodiment according to the present disclosed technology provides a medical service support device, a medical service support method, and a program capable of supporting efficient performance of an endoscope handling service.

A medical service support device according to a first aspect of the present disclosed technology comprises: a processor; and a memory that is connected to or built into the processor, in which the processor is configured to: derive, based on standard operation time, during which a medical device that is used in an endoscope handling service is being operated, and on standby time for an endoscope procedure service included in the endoscope handling service, an operation rate reference value of the medical device; and output medical service support information obtained in accordance with a difference degree between an operation rate of the medical device and the operation rate reference value.

A medical service support method according to a second aspect of the present disclosed technology comprises: deriving, based on standard operation time, during which a medical device that is used in an endoscope handling service is being operated, and on standby time for an endoscope procedure service included in the endoscope handling service, an operation rate reference value of the medical device; and outputting medical service support information obtained in accordance with a difference degree between an operation rate of the medical device and the operation rate reference value.

A program according to a third aspect of the present disclosed technology that causes a computer to execute a process comprising: deriving, based on standard operation time, during which a medical device that is used in an endoscope handling service is being operated, and on standby time for an endoscope procedure service included in the endoscope handling service, an operation rate reference value of the medical device; and outputting medical service support information obtained in accordance with a difference degree between an operation rate of the medical device and the operation rate reference value.

### BRIEF DESCRIPTION OF THE DRAWINGS

Exemplary embodiments of the technology of the disclosure will be described in detail based on the following figures, wherein:

FIG. 1 is a conceptual diagram showing a schematic configuration of a medical service support system;

FIG. 2 is a flowchart showing an example of a flow of an endoscope handling service;

FIG. 3 is a block diagram showing an example of a hardware configuration of an electrical system of an endoscope part management device;

FIG. 4 is a block diagram showing an example of a function of a CPU and a storage content of a storage in an endoscope part management device main body;

FIG. 5 is a conceptual diagram showing an example of a content of an endoscopy management database;

FIG. 6 is a conceptual diagram showing an example of a content of a washer management database;

FIG. 7 is a block diagram showing an example of a hardware configuration of an electrical system of a support device server;

FIG. 8 is a block diagram showing an example of a function of a CPU and a storage content of a storage in the support device server;

FIG. 9 is a block diagram showing an example of a storage content of a management information storage device;

FIG. 10 is a flowchart showing an example of a flow of a management device control process;

FIG. 11 is a flowchart showing an example of a flow of a medical service support process according to a first embodiment;

FIG. 12 is a screen view showing an example of a medical service support screen according to the first embodiment;

FIG. 13 is a screen view showing a modification example of the medical service support screen according to the first embodiment;

FIG. 14 is a conceptual diagram showing a modification example of a content of the endoscopy management database;

FIG. 15 is a flowchart showing an example of a flow of a medical service support process according to a second embodiment;

FIG. 16 is a screen view showing an example of a medical service support screen according to the second embodiment;

FIG. 17 is a screen view showing a modification example of a medical service support screen according to the second embodiment;

FIG. 18 is a flowchart showing an example of a flow of a medical service support process according to a third embodiment;

FIG. 19 is a screen view showing an example of a medical service support screen according to the third embodiment; and

FIG. 20 is a screen view showing a modification example of a medical service support screen according to the third embodiment.

### DETAILED DESCRIPTION

An example of an embodiment of a medical service support device, a medical service support method, and a program according to the present disclosed technology will be described with reference to the accompanying drawings.

### First Embodiment

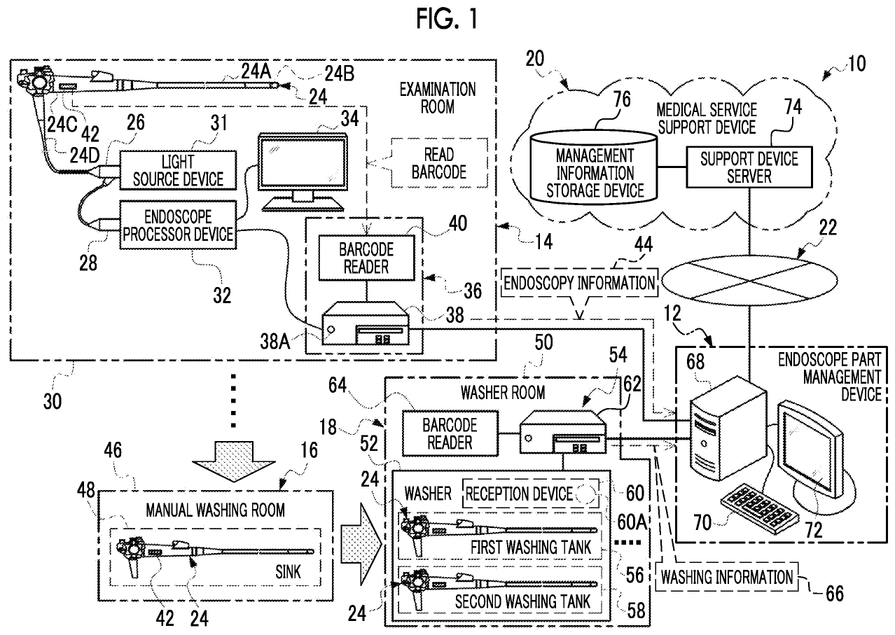

As an example, as shown in FIG. 1, a medical service support system 10 includes an endoscope part management device 12, a plurality of examination room facilities 14, a manual washing facility 16, an automatic washing facility 18, and a medical service support device 20. Here, the medical service support device 20 is an example of a “medical service support device” according to the present disclosed technology.

The medical service support system 10 is a system that supports an endoscope handling service. In the present first embodiment, the endoscope handling service refers to, for example, a service performed by a person engaged in examination (hereinafter, also referred to as an “endoscopy worker”) using an endoscope 24 (hereinafter, also referred to as an “endoscopy”) in a medical installation facility (for example, a hospital). The endoscope handling service includes an endoscopy, a manual washing service, and an automatic washing service. The endoscopy is an example of an “endoscope procedure service”, a “first endoscope procedure service”, and a “second endoscope procedure service” according to the present disclosed technology, and is performed by a doctor or the like.

The medical installation facility has a plurality of endoscopes 24, and a type of endoscope 24, which corresponds to an examination item, is used in each of the plurality of examination room facilities 14. Examples of the type of endoscope 24 include an upper gastrointestinal scope, a pancreatic duct scope, a bile duct scope, a duodenal scope, a colon scope, an intracerebral scope, an otolaryngology scope, a bronchial scope, and the like.

The endoscope 24 includes an insertion part 24A, an imaging part 24B, an operation part 24C, and a universal cord 24D. The insertion part 24A is inserted into a subject.

The imaging part 24B is provided at a distal end part of the insertion part 24A. The imaging part 24B includes a complementary metal oxide semiconductor (CMOS) type imaging element. A charge-coupled device (CCD) type imaging element may be used instead of the CMOS type imaging element.

The operation part 24C is installed to be continuous with a proximal end side of the insertion part 24A, and a doctor holds the operation part 24C and performs various operations. By operating the operation part 24C, the insertion part 24A is bent or imaging is performed by the imaging part 24B.

The universal cord 24D is installed to be continuous with the operation part 24C. The universal cord 24D encompasses a light guide (not shown), a fluid tube (not shown), and a signal cable (not shown). Connectors 26 and 28 are provided at an end part of the universal cord 24D. The connector 28 is branched from the connector 26.

A plurality of examination room facilities 14 are provided in the medical installation facility, and each of the examination room facilities 14 includes an examination room 30, a light source device 31, an endoscope processor device 32, a display 34, and an information processing apparatus 36. The light source device 31, the endoscope processor device 32, the display 34, and the information processing apparatus 36 are installed in the examination room 30.

A plurality of information processing apparatuses 36 are present and are provided in each of a plurality of medical sites where the endoscope handling service is performed. In the present first embodiment, one information processing apparatus 36 is assigned to each of the examination rooms 30. The examination room 30 is an example of a “medical site” according to the present disclosed technology.

The information processing apparatus 36 is an example of an “information processing apparatus” according to the present disclosed technology and is used together with the endoscope 24 in the endoscopy. The information processing apparatus 36 operates every time an endoscopy is performed on one subject in each examination room 30. The information processing apparatus 36 is continuously operated while an endoscopy is performed on one subject. That is, a period from when the operation of the information processing apparatus 36 is started to when the operation of the information processing apparatus 36 is ended corresponds to a period during which an endoscopy is performed on one subject.

The connector 26 is connected to the light source device 31. In a case where the connector 26 is connected to the light source device 31, a light guide and a fluid tube in the universal cord 24D are inserted into the light source device 31. The light source device 31 supplies illumination light, water, and gas to the endoscope 24 via the light guide and the fluid tube. Accordingly, the illumination light is emitted toward a test observation site from an illumination window (not shown) on a distal end surface of the insertion part 24A. Further, water and gas are injected from a nozzle (not shown) on the distal end surface of the insertion part 24A toward the observation window according to the operation performed on the operation part 24C. Here, although an example of the embodiment has been described in which the light source device 31 supplies water and gas to the endoscope 24, this is merely an example, and at least one of water or gas may be supplied to the endoscope 24 from a device other than the light source device.

The connector 28 is connected to the endoscope processor device 32. In a case where the connector 28 is connected to the endoscope processor device 32, a signal cable in the universal cord 24D is electrically connected to the endoscope processor device 32. The endoscope processor device 32 controls the operation of the endoscope 24 by supplying a control signal to the endoscope 24 via a signal cable. Further, the endoscope 24 outputs an imaging signal, which is obtained by causing the imaging part 24B to image the test observation site, to the endoscope processor device 32 via the signal cable.

The endoscope processor device 32 generates a moving image as an image showing the test observation site based on the imaging signal input from the endoscope 24. A frame rate of the moving image is, for example, 30 frames per second (fps). Further, the endoscope processor device 32 generates a still image as an image showing the test observation site in response to an operation performed on the operation part 24C. The endoscope processor device 32 is connected to the display 34 and displays an image, which is generated based on the imaging signal, on the display 34.

In the present first embodiment, although the light source device 31 and the endoscope processor device 32 are separate from the information processing apparatus 36, this is merely an example, and at least one of the light source device 31 and the endoscope processor device 32 may be integrated with the information processing apparatus 36.

The information processing apparatus 36 includes an information processing apparatus main body 38 and a barcode reader 40. The information processing apparatus main body 38 is configured to include a computer including a processor and a storage device. Examples of the processor include a central processing unit (CPU). Examples of the storage device include a combination of a non-volatile memory such as an electrically erasable and programmable read only memory (EEPROM), a solid state drive (SSD), and/or a hard disk drive (HDD), and a volatile memory such as a random access memory (RAM).

An examination start button 38A is provided on the information processing apparatus main body 38. The examination start button 38A is an alternate type push button. Further, the examination start button 38A is turned on at a time of starting an endoscopy for one subject and is turned off at a time of ending an endoscopy on one subject. In a case where the examination start button 38A is turned on, the information processing apparatus main body 38 starts the operation, and in a case where the examination start button 38A is turned off, the information processing apparatus main body 38 stops the operation.

The endoscope part management device 12 is connected to the information processing apparatus main body 38. An information processing apparatus ID, which is an identification (ID) capable of specifying the information processing apparatus main body 38, is assigned to the information processing apparatus main body 38, and the endoscope part management device 12 centrally manages each information processing apparatus main body 38, which is installed in each of the examination rooms 30, by using the information processing apparatus ID.

Further, the endoscope processor device 32 is connected to the information processing apparatus main body 38. That is, the endoscope 24 is connected to the information processing apparatus main body 38 via the endoscope processor device 32. Further, a barcode reader 40 is also connected to the information processing apparatus main body 38.

The information processing apparatus main body 38 is operated under the control of the endoscope part management device 12 and controls the operations of the endoscope processor device 32 and the barcode reader 40. The information processing apparatus main body 38 acquires an image, which is generated based on an imaging signal by the endoscope processor device 32, and outputs the acquired image to the endoscope part management device 12.

A barcode 42 is provided on the endoscope 24. In the example shown in FIG. 1, the barcode 42 is provided on the operation part 24C. The barcode 42 is information in which an endoscope ID that is capable of uniquely specifying the endoscope 24 is encrypted. The barcode 42 is read by the barcode reader 40 in a case where an endoscopy is started. The barcode reader 40 outputs the endoscope ID obtained by reading the barcode 42 to the information processing apparatus main body 38.

The barcode reader 40 is also capable of reading a barcode (not shown) that is uniquely assigned to the subject. The barcode, which is assigned to the subject (hereinafter, also referred to as a “subject barcode”), is information in which a subject ID that is capable of uniquely specifying the subject is encrypted, and is attached to, for example, a band (for example, a wristband) attached to the subject's wrist or the like. The subject barcode is read by the barcode reader 40 at a time at which an endoscopy is started.

The information processing apparatus main body 38 outputs various information related to the subject specified through the subject ID and endoscopy information 44 associated with various IDs to the endoscope part management device 12. Here, the various IDs refer to the endoscope IDs obtained by reading the barcode 42 with the barcode reader 40, and the subject IDs obtained by reading the subject barcode with the barcode reader 40. Here, the endoscope IDs and the subject IDs are exemplified as various IDs, but the present disclosed technology is not limited to this, and only the endoscope IDs may be used.

Further, examples of various types of information related to the subject include, for example, an image generated by the endoscope processor device 32, an examination start time point, an examination end time point, and the like. The information processing apparatus main body 38 includes a clock (for example, a real-time clock) and acquires the examination start time point and the examination end time point from the clock. For example, the examination start time point is a time point when the examination start button 38A is turned on, and the examination end time point is a time point when the examination start button 38A is turned off.

The manual washing facility 16 includes a manual washing room 46 and a sink 48. The sink 48 is installed in the manual washing room 46, and manual washing of the endoscope 24 is performed in the sink 48. Here, the manual washing refers to work in which an endoscopy worker manually and physically removes stains on the endoscope 24 by using a detergent, a brush, a sponge, or the like.

The automatic washing facility 18 includes a washer room 50, a plurality of washers 52, and a washer management device 54. The plurality of washers 52 and the washer management devices 54 are installed in the washer room 50. The washer 52 is a device that mechanically washes the endoscope 24 on which manual washing is performed. The plurality of washers 52 include a single-endoscope washer that is capable of washing only one endoscope 24 and a dual-endoscope washer that is capable of washing two endoscopes 24 together. The single-endoscope washer is also conventionally referred to as a single washer, and the dual-endoscope washer is also conventionally referred to as a dual washer.

The single-endoscope washer is provided with only one washing tank into which one endoscope 24 is inserted, whereas the dual-endoscope washer is provided with a first washing tank 56 and a second washing tank 58. One endoscope 24 is inserted into each of the first washing tank 56 and the second washing tank 58. Hereinafter, in a case where it is not necessary to distinguish among the washing tank of the single-endoscope washer, the first washing tank 56, and the second washing tank 58, a term “washing tank” will be used without reference numerals.

The washer 52 includes a reception device 60. The reception device 60 includes a plurality of buttons and a dial. The plurality of buttons include a washing start button 60A. The washing start button 60A is a button that receives an instruction for causing the washer 52 to start washing the endoscope 24 in the washing tank. In a case where the washing start button 60A is turned on, the washer 52 starts the operation. In a case where the washer 52 is operated, the washer 52 sequentially performs washing, rinsing, dehydration, and the like with a detergent for the endoscope 24 in the washing tank according to a default program.

The washing time by the washer 52, that is, the operation time of the washer 52, is designated by operating the dial included in the reception device 60. After a lapse of time designated by operating the dial, the washer 52 stops the operation.

The washer management device 54 includes a washer management device main body 62 and a barcode reader 64. The washer management device main body 62 is configured to include a computer including a processor and a storage device. Examples of the processor include a CPU. Examples of the storage device include a combination of a non-volatile memory such as an EEPROM, an SSD, and/or an HDD, and a volatile memory such as a RAM.

The plurality of washers 52 are connected to the washer management device main body 62, and the washer management device main body 62 centrally manages the plurality of washers 52. For example, the washer management device main body 62 includes a clock and acquires a washing start time point and a washing end time point of each of the plurality of washers 52 from the clock. The washing start time point is a time point when the washing start button 60A of the washer 52 is turned on, and the washing end time point is a time point when the operation of the washer 52 is stopped (for example, a time point when washing of the endoscope 24 in the washing tank is ended).

Here, although an example of the embodiment has been described in which the plurality of washers 52 are centrally managed by the washer management device 54, this is merely an example, and each of a plurality of washer management devices 54 may individually manage the plurality of washers 52, and one washer management device 54 may centrally manage the plurality of washers 52.

Further, here, although a time point when the washing start button 60A is turned on is exemplified as the washing start time point, this is merely an example, and, for example, the washing start time point may be determined according to an instruction given to a device other than the washer management device 54 such as the endoscope part management device 12. Further, here, although a time point when the operation of the washer 52 is stopped is exemplified as the washing end time point, this is merely an example, and the washing end time point may be a time point when a timer for managing the washing time is ended, or a time point when the washing start button 60A is turned off.

A washer ID, which is an ID that is capable of specifying the washer 52, is assigned to each of the plurality of washers 52, and the washer management device main body 62 centrally manages the plurality of washers 52 by using washer IDs.

The barcode reader 64 is connected to the washer management device main body 62. The endoscope part management device 12 is connected to the washer management device main body 62, and the washer management device main body 62 is operated under the control of the endoscope part management device 12 and controls the operation of the plurality of washers 52 and the barcode reader 64.

The barcode 42 of the endoscope 24, on which manual washing is performed, is read by the barcode reader 64. The barcode reader 64 outputs the endoscope ID obtained by reading the barcode 42 to the washer management device main body 62.

The washer management device main body 62 outputs various types of information related to the washer 52 and washing information 66 associated with the endoscope ID corresponding to the endoscope 24 washed by the washer 52 to the endoscope part management device 12. Here, examples of various types of information related to the washer 52 include a washer ID, washing start time point, and washing end time point.

Each of the endoscope 24, the information processing apparatus 36, and the washer 52 is an example of a “medical device used in the endoscope handling service” according to the present disclosed technology. Further, in the following, for convenience of explanation, in a case where it is not necessary to distinguish among the endoscope 24, the information processing apparatus 36, and the washer 52, a term “medical device” will also be used without reference numerals.

The endoscope part management device 12 is a device that manages a part (endoscope part) that is responsible for the endoscope handling service in a medical installation facility. The endoscope part management device 12 includes an endoscope part management device main body 68, a reception device 70, and a display 72. The display 72 is an example of a “presentation device” according to the present disclosed technology.

As described in detail later, the endoscope part management device main body 68 is a device configured to include a computer. The reception device 70 is connected to the endoscope part management device main body 68. The reception device 70 includes a keyboard, a mouse, a touch panel, and/or the like, and receives an instruction from a user or the like of the endoscope part management device 12. The display 72 is connected to the endoscope part management device main body 68. The display 72 displays various types of information under the control of the endoscope part management device main body 68. Examples of the display 72 include an electro-luminescence (EL) display, a liquid crystal display, and the like.

The endoscope part management device main body 68 acquires the endoscopy information 44 output from the information processing apparatus main body 38 and the washing information 66 output from the washer management device main body 62, and executes a process by using the acquired endoscopy information 44 and washing information 66. In the following, for convenience of explanation, in a case where it is not necessary to distinguish between the endoscopy information 44 and the washing information 66, a term “management target information” will be used without reference numerals.

In the present first embodiment, the endoscope part management device 12 is connected to the medical service support device 20 via a network 22. The network 22 is, for example, the Internet. Note that the network 22 is not limited to the Internet and may be a wide area network (WAN) and/or a local area network (LAN) such as an intranet. Further, the endoscope part management device 12 and the medical service support device 20 may be integrally formed, and the medical service support device 20 may have at least some of the functions of the endoscope part management device 12.

The endoscope part management device main body 68 exchanges information with the medical service support device 20 via the network 22. The medical service support device 20 is realized by cloud computing. Here, although cloud computing is exemplified, this is merely an example, and, for example, the medical service support device 20 may be implemented by a mainframe or implemented by network computing such as fog computing, edge computing, or grid computing.

The medical service support device 20 includes a support device server 74 and a management information storage device 76. The management information storage device 76 is connected to the support device server 74. The support device server 74 is connected to the endoscope part management device main body 68 via the network 22, performs a process in response to a request from the endoscope part management device main body 68, and provides a processing result to the endoscope part management device main body 68.

The endoscope part management device main body 68 anonymizes the endoscopy information 44 and transmits the anonymized endoscopy information 44 and washing information 66 to the support device server 74. The anonymization of the endoscopy information 44 means the anonymization of the subject specified based on the subject ID included in the endoscopy information 44.

The support device server 74 receives the endoscopy information 44 and the washing information 66, and stores the received endoscopy information 44 and the washing information 66 in the management information storage device 76. The support device server 74 selectively acquires the endoscopy information 44 and the washing information 66 from the management information storage device 76 as necessary, and executes a process by using the acquired information.

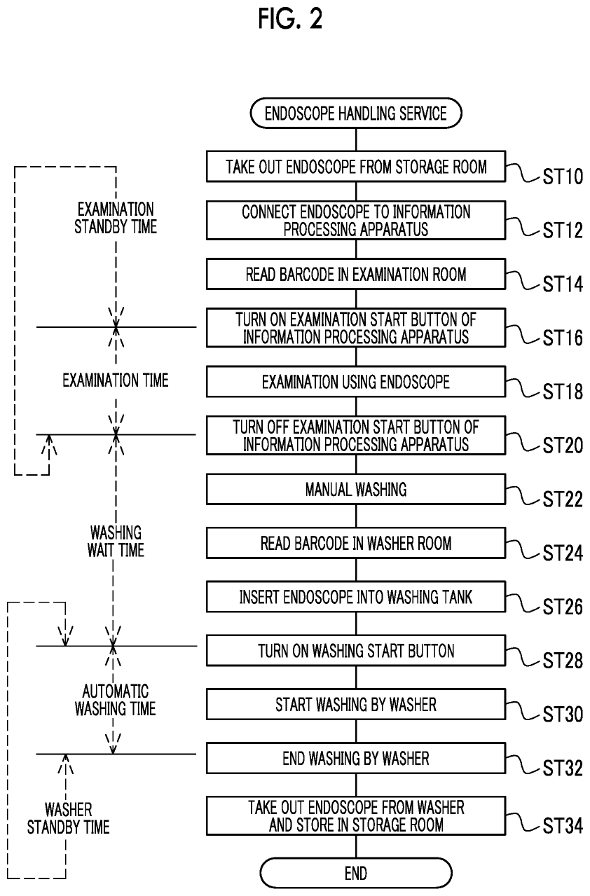

FIG. 2 shows an example of a flow of an endoscope handling service performed by the endoscopy worker. In the following, for convenience of explanation, the description is based on the premise that the usable endoscope 24 (for example, the endoscope 24 washed by the washer 52) is stored in a storage room (not shown) in a medical installation facility by the endoscopy worker.

In the endoscope handling service shown in FIG. 2, first, in step ST10, the endoscope 24 is taken out from the storage room by the endoscopy worker, and the endoscope 24 is transported into one examination room 30 among the plurality of examination rooms 30.

In the next step ST12, the endoscope 24 is connected to the information processing apparatus 36 in the examination room 30 via the light source device 31 and the endoscope processor device 32.

In the next step ST14, the barcode 42 of the endoscope 24 is read by the barcode reader 40 in the examination room 30. Accordingly, the endoscope ID of the endoscope 24 is acquired by the information processing apparatus 36.

In the next step ST16, the examination start button 38A of the information processing apparatus 36 is turned on. Further, an examination start button (not shown) is also provided in the endoscope processor device 32, and the examination start button of the endoscope processor device 32 is turned on. Accordingly, an endoscopy is started. Here, although an example of the embodiment has been described in which a process of step ST14 is performed prior to a process of step ST16, this is merely an example, and the process of step ST16 may be performed prior to the process of step ST14. That is, the order of step ST14 and step ST16 may be reversed.

In the following, for convenience of explanation, although the description is based on the premise that an endoscopy is started on a condition that the examination start button 38A is turned on, the present disclosed technology is not limited to this. For example, the endoscope processor device 32 may also include an examination start button similar to the examination start button 38A of the information processing apparatus 36, and the endoscopy may be started on a condition that the examination start button 38A of the information processing apparatus 36 is turned on and the examination start button of the endoscope processor device 32 is also turned on. In this case, the endoscope processor device 32 and the information processing apparatus 36 are examples of the “information processing apparatuses” according to the present disclosed technology.

In the next step ST18, an examination using the endoscope 24 (such as imaging of a test observation site by the imaging part 24B of the endoscope 24) is performed by a doctor or the like. Accordingly, the information processing apparatus 36 generates an image based on an imaging signal obtained by imaging by the imaging part 24B.

In the next step ST20, the examination start button 38A of the information processing apparatus 36 is turned off. Further, the examination start button of the endoscope processor device 32 is also turned off. Accordingly, the endoscopy is ended. The time during which the information processing apparatus 36 is being operated corresponds to time from when the examination start button 38A is turned on in step ST16 to when the examination start button 38A is turned off in step ST20, and is time (hereinafter, also referred to as “examination time”) during which the endoscopy is being performed.

In the following, for convenience of explanation, although the description is based on the premise that the endoscopy is ended on a condition that the examination start button 38A is turned off, the present disclosed technology is not limited to this. For example, the endoscopy may end on a condition that the examination start button 38A of the information processing apparatus 36 is turned off and the examination start button of the endoscope processor device 32 is also turned off.

Note that the examination time is an example of “operation time of the information processing apparatus” according to the present disclosed technology, and the time from when the examination start button 38A is turned on in step ST16 to when the examination start button 38A is turned off in step ST20 corresponds to “time from when a first endoscope procedure service using the information processing apparatus is started to when the first endoscope procedure service is ended” according to the present disclosed technology.

In the next step ST22, manual washing of the endoscope 24 used in step ST18 is performed. In the next step ST24, the barcode 42 of the endoscope 24 manually washed in the washer room 50 is read by the barcode reader 64. Accordingly, the endoscope ID of the endoscope 24 is acquired by the washer management device 54.

In the next step ST26, the endoscope 24 is inserted into the washing tank of the washer 52. In the next step ST28, the washing start button 60A is turned on.

The time from when the examination start button 38A is turned off in step ST20 to when the washing start button 60A is turned on in step ST28 is time corresponding to time during which the manual washing is being performed (hereinafter, also referred to “manual washing time”).

In the next step ST30, washing of the endoscope 24, which is inserted into the washing tank in step ST26, is started by the washer 52. In the next step ST32, after a lapse of designated time after the washing start button 60A is turned on in step ST28, the washing of the endoscope 24, which is inserted into the washing tank in step ST26, by the washer 52 is ended, and one endoscope handling service is ended.

The time from when the washing start button 60A is turned on in step ST28 to when the washing of the endoscope 24 by the washer 52 is ended in step ST32 is time corresponding to time during which washing of the endoscope 24 is being performed by the washer 52 (hereinafter, also referred to as “automatic washing time”).

In the next step ST34, the endoscope 24 is taken out from the washer 52, and the taken-out endoscope 24 is stored in the storage room.

In a case where N is a natural number, in a specific examination room 30, time required from immediately before a time when an N-th endoscopy is ended (immediately before a time when the examination start button 38A is turned off in step ST20 included in the N-th endoscope handling service) to immediately before a time when an (N+1)-th endoscopy is started (immediately before a time when the examination start button 38A is turned on in step ST16 included in the (N+1)-th endoscope handling service) is time corresponding to time during which the endoscopy is on standby in a specific examination room 30 (hereinafter, also referred to as “examination standby time”). The examination standby time is an example of “standby time” according to the present disclosed technology.

Further, in the washer room 50, time that is required from when the N-th washing by the washer 52 is ended to when the (N+1)-th washing by the washer 52 is started (restarted), is time corresponding to time during which the washer 52 is on standby for washing of the endoscope 24 (hereinafter, also referred to as “washer standby time”). Here, a target to be washed by the washer 52 is an unspecified endoscope 24. However, this is merely an example, and the specific endoscope 24 may be washed by the specific washer 52.

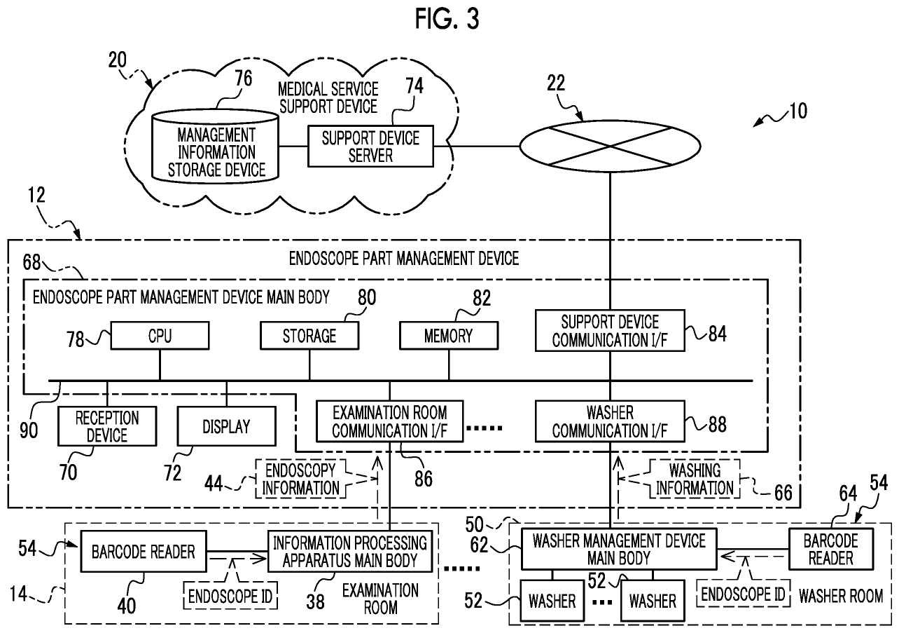

As an example, as shown in FIG. 3, the endoscope part management device main body 68 includes a CPU 78, a storage 80, a memory 82, a support device communication interface (I/F) 84, a plurality of examination room communication I/Fs 86, and a washer communication I/F 88. The CPU 78, the storage 80, the memory 82, the support device communication I/F 84, the plurality of examination room communication I/Fs 86, and the washer communication I/F 88 are connected to a bus 90.

The CPU 78 controls the entire endoscope part management device main body 68. The storage 80 is a non-volatile storage device that stores various programs, various parameters, and the like. Examples of the storage 80 include an EEPROM, an SSD, and/or an HDD. The memory 82 is a memory in which information is temporarily stored and is used as a work memory by the CPU 78. Examples of the memory 82 include a RAM.

The support device communication I/F 84 is connected to the network 22 and controls the exchange of information between the CPU 78 and the support device server 74. For example, the support device communication I/F 84 transmits information in response to a request from the CPU 78 to the support device server 74 via the network 22, receives the information transmitted from the support device server 74, and outputs the received information to the CPU 78.

The examination room communication I/F 86 is provided for each examination room 30 and is connected to the information processing apparatus main body 38 in the examination room 30. The examination room communication I/F 86 controls the exchange of information between the CPU 78 and the information processing apparatus main body 38. For example, the examination room communication I/F 86 transmits information in response to a request from the CPU 78 to the information processing apparatus main body 38 designated by the CPU 78 among the plurality of information processing apparatus main bodies 38. Further, the examination room communication I/F 86 acquires the endoscopy information 44 from the information processing apparatus main body 38 and outputs the acquired endoscopy information 44 to the CPU 78.

The washer communication I/F 88 is connected to a washer management device main body 62 and controls the exchange of information between the CPU 78 and the washer management device main body 62. For example, the washer communication I/F 88 transmits information in response to a request from the CPU 78 to the washer management device main body 62. Further, the washer communication I/F 88 acquires the washing information 66 from the washer management device main body 62 and outputs the acquired washing information 66 to the CPU 78.

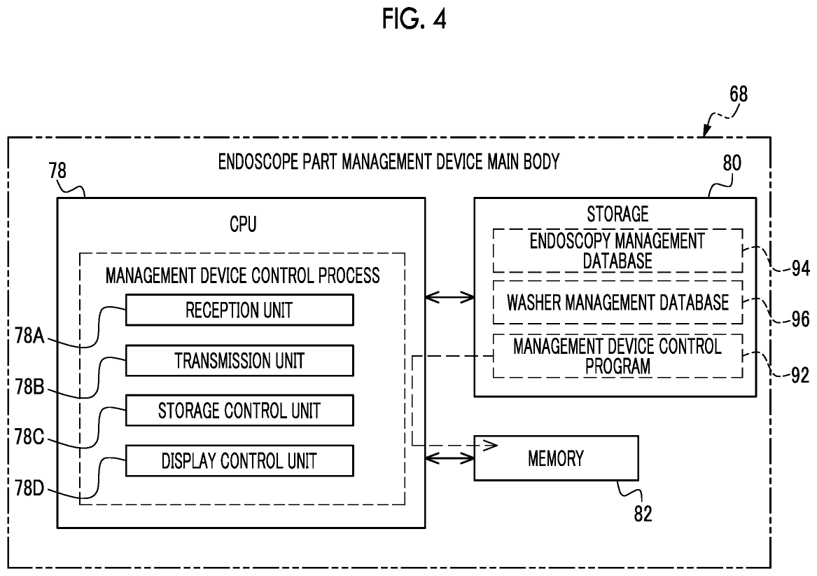

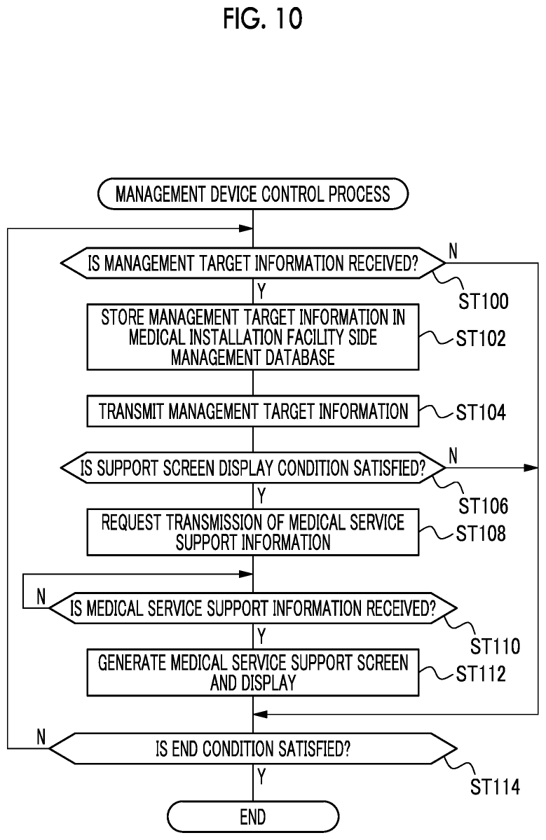

As an example, as shown in FIG. 4, in the endoscope part management device main body 68, the storage 80 stores a management device control program 92. The CPU 78 reads the management device control program 92 from the storage 80 and performs a management device control process by executing the read management device control program 92 on the memory 82 (see FIG. 10). The management device control process is realized by the CPU 78 operating as a reception unit 78A, a transmission unit 78B, a storage control unit 78C, and a display control unit 78D in accordance with the management device control program 92. Specific processing contents of the reception unit 78A, the transmission unit 78B, the storage control unit 78C, and the display control unit 78D will be described later with reference to FIG. 10.

An endoscopy management database 94 and a washer management database 96 are constructed in the storage 80. The storage control unit 78C acquires the endoscopy information 44 (see FIGS. 1 and 3) via the examination room communication I/F 86 (see FIG. 3) and stores the acquired endoscopy information 44 in the endoscopy management database 94. Further, the storage control unit 78C acquires the washing information 66 (see FIGS. 1 and 3) via the washer communication I/F 88 (see FIG. 3) and stores the acquired washing information 66 in the washer management database 96. In the following, for convenience of explanation, in a case where it is not necessary to distinguish between the endoscopy management database 94 and the washer management database 96, a term “medical installation facility side management database” will also be used.

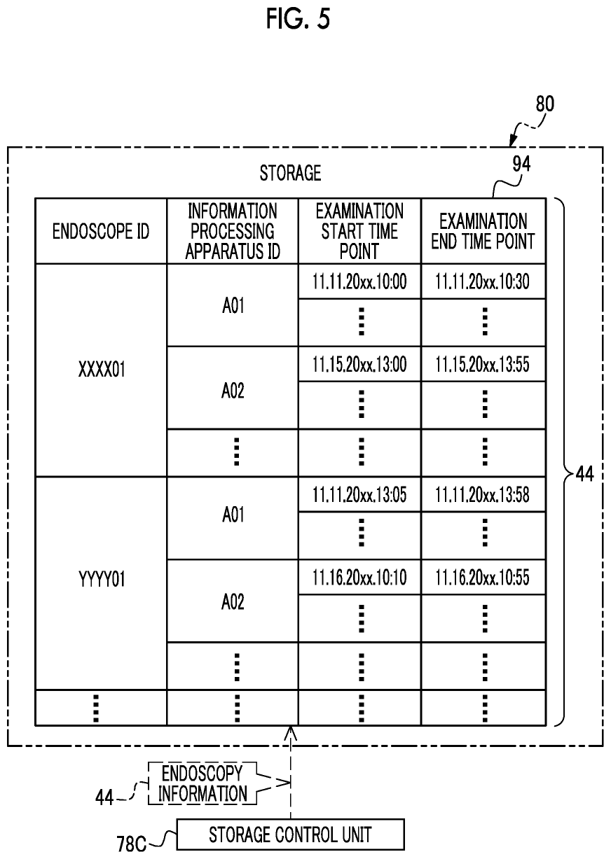

As an example, as shown in FIG. 5, in the endoscopy management database 94, the endoscopy information 44 acquired by the storage control unit 78C from each information processing apparatus main body 38 installed in each of the plurality of examination rooms 30 is stored. In the endoscopy management database 94, the examination start time point and the examination end time point are associated for each endoscope ID and for each information processing apparatus ID. Further, the subject ID is associated with the examination start time point or the examination end time point (not shown).

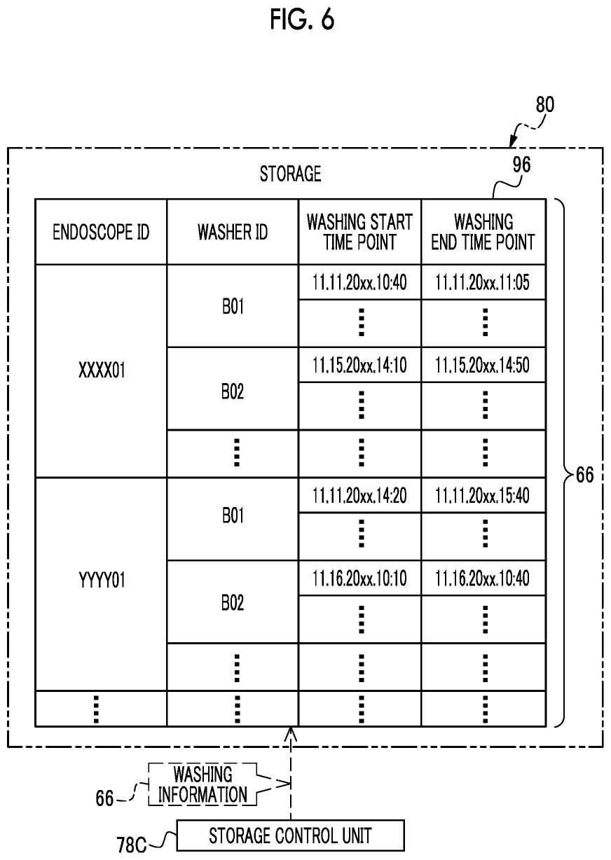

As an example, as shown in FIG. 6, in the washer management database 96, the washing information 66 acquired by the storage control unit 78C from the washer management device main body 62 is stored. In the washer management database 96, the washing start time point and the washing end time point are associated for each endoscope ID and for each washer ID.

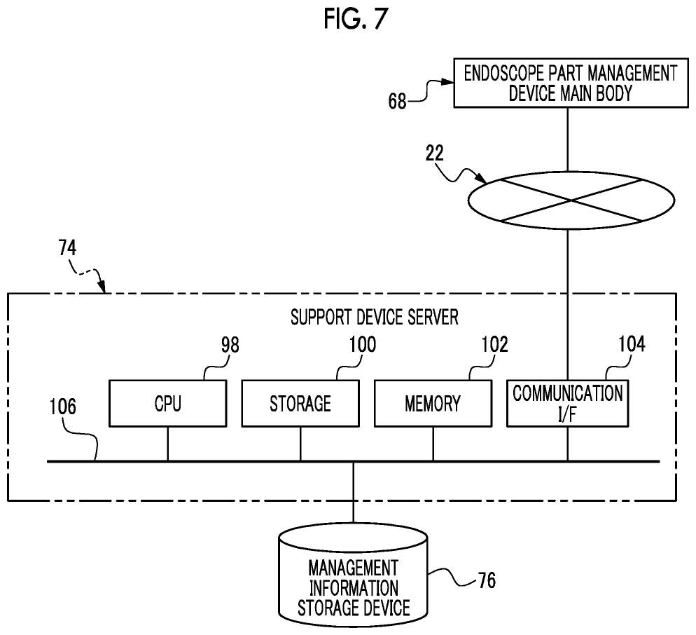

As an example, as shown in FIG. 7, the support device server 74 includes a CPU 98, a storage 100, a memory 102, and a communication I/F 104. The CPU 98 is an example of a “processor” according to the present disclosed technology, and the memory 102 is an example of a “memory” according to the present disclosed technology.

The CPU 98, the storage 100, the memory 102, and the communication I/F 104 are connected to a bus 106. Further, the management information storage device 76 is connected to the bus 106. Examples of the management information storage device 76 include an EEPROM, an SSD, and/or an HDD. In the management information storage device 76, anonymized management target information is stored.

The CPU 98 controls the entire support device server 74. The storage 100 is a non-volatile storage device that stores various programs, various parameters, and the like. Examples of the storage 100 include an EEPROM, an SSD, and/or an HDD. The memory 102 is a memory in which information is temporarily stored and is used as a work memory by the CPU 98. Examples of the memory 102 include a RAM.

The communication I/F 104 is connected to the endoscope part management device main body 68 via the network 22 and controls the exchange of information between the CPU 98 and the endoscope part management device main body 68. For example, the communication I/F 104 receives the information transmitted from the endoscope part management device main body 68 and outputs the received information to the CPU 78. Further, the communication I/F 104 transmits information in response to a request from the CPU 98 to the endoscope part management device main body 68 via the network 22.

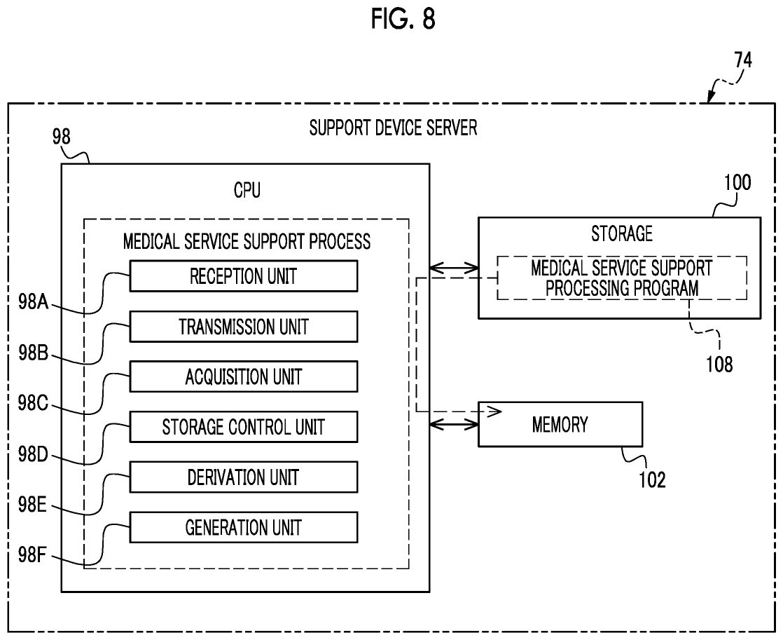

As an example, as shown in FIG. 8, in the support device server 74, the storage 100 stores a medical service support processing program 108. The support device server 74 is an example of a “computer” according to the present disclosed technology, and the medical service support processing program 108 is an example of a “program” according to the present disclosed technology.

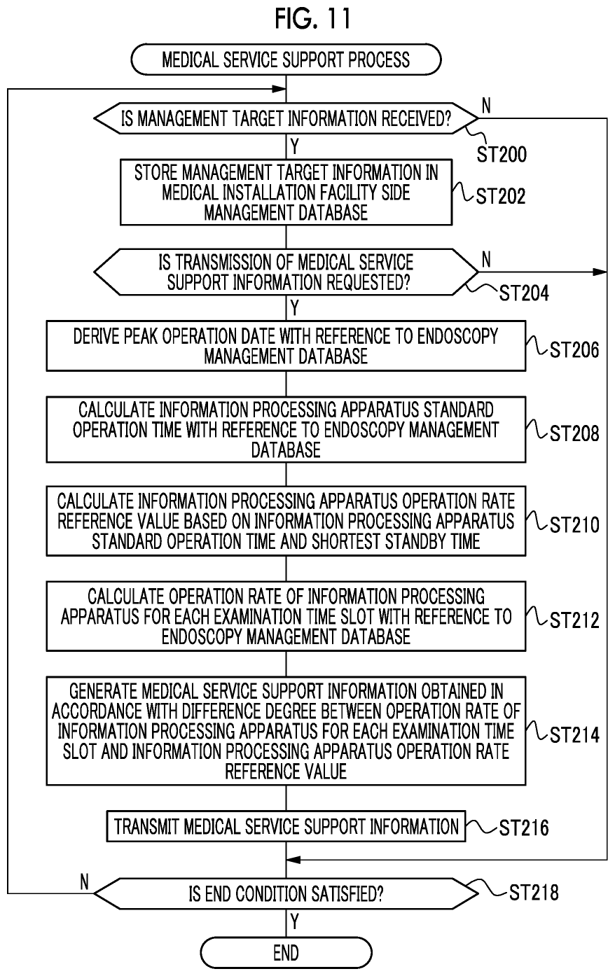

The CPU 98 reads the medical service support processing program 108 from the storage 100 and performs a medical service support process by executing the read medical service support processing program 108 on the memory 102 (see FIG. 11). The medical service support process is realized by the CPU 98 operating as a reception unit 98A, a transmission unit 98B, an acquisition unit 98C, a storage control unit 98D, a derivation unit 98E, and a generation unit 98F in accordance with the medical service support processing program 108. Specific processing contents of the reception unit 98A, the transmission unit 98B, the acquisition unit 98C, the storage control unit 98D, the derivation unit 98E, and the generation unit 98F will be described later with reference to FIG. 11.

By performing the medical service support process, and based on the standard operation time, during which the medical device is being operated, and on the standby time for endoscopy, the CPU 98 derives an operation rate reference value of the medical device and outputs medical service support information obtained in accordance with a difference degree between an operation rate of the medical device and the operation rate reference value. Hereinafter, the operation rate of the medical device is also simply referred to as an “operation rate”, and the operation rate reference value of the medical device is also simply referred to as an “operation rate reference value”.

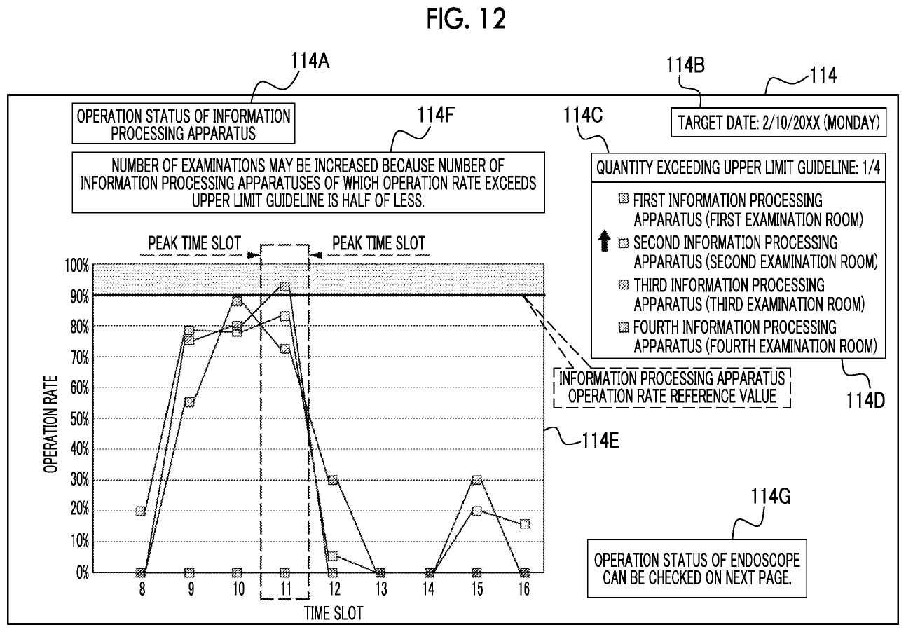

As described in detail later, the medical service support information is information that includes medical device related information related to the medical device of which the operation rate is within an operation rate range defined based on the operation rate reference value. Further, the medical device related information is information that includes information related to the medical device of which the operation rate is within the operation rate range during a time period when an operation degree of the medical device is equal to or higher than a first reference degree. Here, the time period when the operation degree of the medical device is equal to or higher than the first reference degree refers to a time period when a value based on the number of endoscopies is equal to or greater than a default value. In the present first embodiment, the medical service support information includes screen information indicating a medical service support screen 114 (see FIG. 12) as the medical device related information.

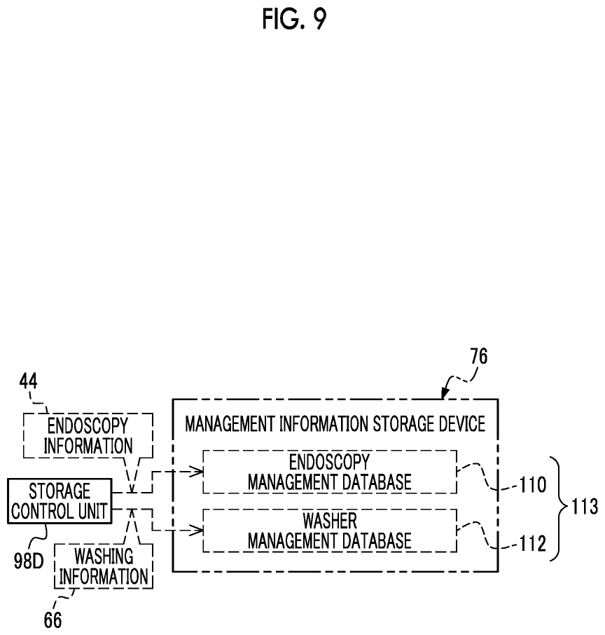

As an example, as shown in FIG. 9, a support device management database 113 is constructed in the management information storage device 76. The support device management database 113 includes an endoscopy management database 110 and a washer management database 112. The anonymized endoscopy information 44 is stored in the endoscopy management database 110, and the washing information 66 is stored in the washer management database 112.

The storage control unit 98D acquires the anonymized endoscopy information 44 from the endoscope part management device main body 68 via the communication I/F 104 (see FIG. 7) and stores the acquired endoscopy information 44 in the endoscopy management database 110. The storage control unit 98D acquires the washing information 66 (see FIG. 6) from the endoscope part management device main body 68 via the communication I/F 104 (see FIG. 7) and stores the acquired washing information 66 in the washer management database 112.

Next, the operation of the medical service support system will be described.

FIG. 10 shows an example of a flow of a management device control process performed by the CPU 78 of the endoscope part management device 12. In the present first embodiment, the information processing apparatus 36 will be described below as an example of the medical device.

In the management device control process shown in FIG. 10, first, in step ST100, the reception unit 78A determines whether or not the management target information is received through the examination room communication I/F 86 (see FIG. 3) or the washer communication I/F 88 (see FIG. 3). In step ST100, in a case in which the management target information is not received through the examination room communication I/F 86 or the washer communication I/F 88, a negative determination is made, and the management device control process proceeds to step ST114. In step ST100, in a case in which the management target information is received through the examination room communication I/F 86 or the washer communication I/F 88, a positive determination is made, and the management device control process proceeds to step ST102.

In step ST102, the storage control unit 78C stores the management target information, which is received through the examination room communication I/F 86 or the washer communication I/F 88 in step ST100, in the medical installation facility side management database. That is, the endoscopy information 44 is stored in the endoscopy management database 94 (see FIGS. 4 and 5), and the washing information 66 is stored in the washer management database 96 (see FIGS. 4 and 6). After the process in step ST102 is executed, the management device control process proceeds to step ST104.

In step ST104, the transmission unit 78B transmits the management target information, which is received in step ST100, to the support device server 74 via the support device communication I/F 84 (see FIG. 3).

After the process in step ST104 is executed, the management device control process proceeds to step ST106.

In step ST106, the transmission unit 78B determines whether or not a support screen display condition, which is a condition for displaying the medical service support screen 114 (see FIG. 12) on the display 72 (see FIGS. 1 and 3), is satisfied. A first example of the support screen display condition includes a condition that the reception device 70 (see FIGS. 1 and 3) receives an instruction for displaying the medical service support screen 114 on the display 72. Further, a second example of the support screen display condition includes a condition that a time point (for example, 22:00), which is designated in advance, has arrived. Further, the second example of the support screen display condition includes a condition that an amount of information of the management target information stored in the medical installation facility side management database reaches a default amount of information.

In step ST106, in a case where the support screen display condition is not satisfied, a negative determination is made, and the management device control process proceeds to step ST114. In step ST106, in a case where the support screen display condition is satisfied, a positive determination is made, and the management device control process proceeds to step ST108.

In step ST108, the transmission unit 78B requests transmission of the medical service support information from the support device server 74 via the support device communication I/F 84 (see FIG. 3). After the process in step ST108 is executed, the management device control process proceeds to step ST110.

In a case where the process of step ST108 is executed, the processes of step ST206 to step ST216, which are included in the medical service support process shown in FIG. 11, are executed by the support device server 74, and the medical service support information is transmitted from the support device server 74 to the endoscope part management device 12 via the network 22 by executing step ST216.

In step ST110, the reception unit 78A determines whether or not the medical service support information, which is transmitted from the support device server 74, is received through the support device communication I/F 84 (see FIG. 3). In step ST110, in a case where the medical service support information is not received through the support device communication I/F 84, a negative determination is made, and the determination in step ST110 is performed again. In step ST110, in a case in which the medical service support information is received through the support device communication I/F 84, a positive determination is made, and the management device control process proceeds to step ST112.

In step ST112, the display control unit 78D generates the medical service support screen 114 (see FIG. 12) by using the medical service support information received through the support device communication I/F 84 in step ST110 and displays the medical service support screen 114 (see FIG. 12) on the display 72 (see FIGS. 1 and 3). After the process in step ST112 is executed, the management device control process proceeds to step ST114.

In step ST114, the display control unit 78D determines whether or not a condition for ending the management device control process (hereinafter, referred to as a “management device control process end condition”) is satisfied. Examples of the management device control process end condition include a condition that an instruction for ending the management device control process is received by the reception device 70 (see FIGS. 1 and 3).

In step ST114, in a case where the management device control process end condition is not satisfied, a negative determination is made, and the management device control process proceeds to step ST100. In step ST114, in a case where the management device control process end condition is satisfied, the management device control process is ended.

FIG. 11 shows an example of a flow of the medical service support process performed by the CPU 98 of the support device server 74.

In the medical service support process shown in FIG. 11, first, in step ST200, the reception unit 98A determines whether or not the management target information, which is transmitted by executing the process of step ST100 included in the management device control process shown in FIG. 10, is received through the communication I/F 104 (see FIG. 7). In step ST200, in a case where the management target information is not received through the communication I/F 104, a negative determination is made, and the medical service support process proceeds to step ST218. In step ST200, in a case where the management target information is received through the communication I/F 104, a positive determination is made, and the medical service support process proceeds to step ST202.

In step ST202, the storage control unit 98D stores the management target information, which is received through the communication I/F 104 in step ST200, in the support device management database 113. That is, the endoscopy information 44 is stored in the endoscopy management database 110 (see FIG. 9), and the washing information 66 is stored in the washer management database 112 (see FIG. 9). After the process in step ST202 is executed, the medical service support process proceeds to step ST204.

In step ST204, the reception unit 98A determines whether or not transmission of the medical service support information is requested from the endoscope part management device 12 by executing the process of step ST108 included in the management device control process shown in FIG. 10. In step ST204, in a case where the transmission of the medical service support information is not requested from the endoscope part management device 12, a negative determination is made, and the medical service support process proceeds to step ST218. In step ST204, in a case where the transmission of the medical service support information is requested from the endoscope part management device 12, a positive determination is made, and the medical service support process proceeds to step ST206.

In step ST206, the derivation unit 98E specifies a date on which an operation status of the medical device peaks (hereinafter, also referred to as a “peak operation date”) with reference to the endoscopy management database 110 (see FIG. 9). In the present first embodiment, the peak operation date for the medical device refers to a date on which the operation statuses of all the information processing apparatuses 36 (hereinafter also referred to as “all information processing apparatuses 36”) installed in all the examination rooms 30 peak. The peak operation date is an example of “a time period when the operation degree of the medical device is equal to or higher than the first reference degree” and “a time period when the operation degree of the medical device is equal to or higher than the second reference degree” according to the technology of the present disclosure. The operation degree of the medical device indicates information that indicates a ratio of operation of the medical device that can be acquired by the medical service support device and is indicated by, for example, a value based on the number of endoscopies. The value based on the number of endoscopies indicates, for example, the number of endoscopies for each predetermined period such as for each day or for each hour. Further, the value based on the number of endoscopies indicates a total number of endoscopies, which are executed by the plurality of medical devices, for each predetermined period in a case where there are a plurality of medical devices that can be acquired by the medical service support device. The value based on the number of endoscopies may be obtained from the information of the examination start time point and the examination end time point of each endoscopy with reference to the endoscopy management database. The first reference degree or the second reference degree is a default value. More specifically, it is a default value for determining a period during which endoscopy is intensively performed, such as the peak operation date or the peak operation time. The default value may be a fixed value set in advance, or a variable value changed according to a user input. Further, the default value may be a variation value calculated based on the number of endoscopies performed in the past. The default value may be, for example, an average value of the number of endoscopies performed in the past or may be the maximum value of the number of endoscopies performed in the past or a value obtained by adding or multiplying the maximum value by a predetermined value. Further, in a case where the operation degree of the medical device indicates the number of endoscopies for each first period (for example, one day), the highest value, which is within the number of endoscopies for each first period in a second period (for example, one week) longer than the first period, may be defined. The default value indicating the second reference degree may be the same as or different from the default value indicating the first reference degree.

Here, a date on which an endoscopy is performed the highest number of times is regarded as the peak operation date. The date on which an endoscopy is performed the highest number of times is an example of “a time period when a value based on the number of endoscope procedure services is equal to or greater than a default value” according to the present disclosed technology.

The date on which an endoscopy is performed can be specified by using the examination start time point or the examination end time point of the endoscopy information 44 stored in the endoscopy management database 110. Therefore, in the present step ST206, the date on which an endoscopy is performed the highest number of times is specified by the derivation unit 98E by using the examination start time point or the examination end time point of the endoscopy information 44 included in the endoscopy management database 110.

The peak operation date may be specified for all periods that can be specified based on the endoscopy information 44 stored in the endoscopy management database 110 or may be a period (for example, a designated number of years, a designated number of months, or a designated number of days) designated based on the endoscopy information 44 stored in the endoscopy management database 110. The designation of the period is performed, for example, by a user or the like via the reception device 70 (see FIGS. 1 and 3) of the endoscope part management device 12.

In the next step ST208, the derivation unit 98E calculates information processing apparatus standard operation time for the peak operation date derived in step ST206 with reference to the endoscopy management database 110 (see FIG. 9). After the process in step ST208 is executed, the medical service support process proceeds to step ST210.

In step ST208, the information processing apparatus standard operation time is a statistical value of time based on the operation time of the information processing apparatus 36 on the peak operation date, that is, operation time (operation time of all the information processing apparatuses 36, which is examination time for endoscopy performed in all the examination rooms 30 (see FIG. 2)) of all the information processing apparatuses 36 on the peak operation date. For example, the information processing apparatus standard operation time is an average value of operation time of all the information processing apparatuses 36 on the peak operation date. The operation time of the information processing apparatus 36 is specified based on the examination start time point and the examination end time point of the endoscopy information 44 stored in the endoscopy management database 110.

Here, although the average value of the operation time of all the information processing apparatuses 36 is exemplified as the information processing apparatus standard operation time, this is merely an example, and it may be a statistical value such as a median value, the most frequent value, the maximum value, the minimum value, or a percentile of the operation time of all the information processing apparatuses 36. Further, the information processing apparatus standard operation time may be calculated within a time range (for example, within a range of substantially 1 minute to 10 minutes) predetermined with an upper limit value and a lower limit value.

In step ST210, the derivation unit 98E calculates an information processing apparatus operation rate reference value based on the information processing apparatus standard operation time calculated in step ST208 and shortest examination standby time (for example, time designated in advance between 1 minute and 3 minutes). After the process in step ST210 is executed, the medical service support process proceeds to step ST212.

In step ST210, the shortest examination standby time refers to the shortest examination standby time until the information processing apparatus 36 is used. The shortest examination standby time is, for example, predetermined time as the shortest time of the examination standby time shown in FIG. 2. The shortest examination standby time may be a fixed value or a variable value that is changed according to an instruction received by the reception device 70 or the like.

The information processing apparatus operation rate reference value is a value based on a ratio of the information processing apparatus standard operation time with respect to the time including the information processing apparatus standard operation time calculated in step ST208 and the shortest examination standby time. Examples of the information processing apparatus operation rate reference value include a value obtained by multiplying the ratio of the information processing apparatus standard operation time with respect to the sum of the information processing apparatus standard operation time calculated in step ST208 and the shortest examination standby time, by an adjustment factor. Examples of the adjustment factor include 0.8. The adjustment factor of “0.8” is merely an example, and the adjustment factor may be another decimal number less than 1. Further, the adjustment factor may be a fixed value or a variable value.

Further, although an example of the embodiment has been described in which the ratio of the information processing apparatus standard operation time with respect to the sum of the information processing apparatus standard operation time and the shortest examination standby time is multiplied by the adjustment factor, this is merely an example, and a value which is obtained by adding or subtracting an adjustment value to the ratio of the information processing apparatus standard operation time with respect to the sum of the information processing apparatus standard operation time and the shortest examination standby time may be used as the information processing apparatus operation rate reference value.

In step ST212, the derivation unit 98E derives an operation rate of each information processing apparatus 36 for each examination time slot on the peak operation date derived in step ST206 with reference to the endoscopy management database 110. After the process in step ST212 is executed, the medical service support process proceeds to step ST214.

Here, the examination time slot on the peak operation date is a time slot on which an endoscopy is performed on the peak operation date and refers to a time slot from 9:00 to 16:00 in units of one hour. The time slot from 9:00 to 16:00 in units of one hour is merely an example, and may be, for example, a time slot of several minutes to several hours within the time of outpatient treatment at a medical installation facility. Further, the examination time slot on the peak operation date may be a fixed value or a variable value that is changed according to an instruction received by the reception device 70 or the like.

The operation rate of the information processing apparatus 36 is calculated by the derivation unit 98E according to the expression “(operation time of information processing apparatus 36)/{(operation time of information processing apparatus 36)+(examination standby time)}” for each examination time slot on the peak operation date.

In step ST214, the generation unit 98F generates the medical service support information obtained in accordance with a difference degree between the operation rate of each information processing apparatus 36 for each examination time slot on the peak operation date (the operation rate calculated in step ST212) and the information processing apparatus operation rate reference value calculated in step ST210. After the process in step ST214 is executed, the medical service support process proceeds to step ST216.

The medical service support information is information that is capable of being represented via a comment, a figure, or the like by a presentation device (here, the display 72 as an example) and includes screen information indicating the medical service support screen 114 (see FIG. 12). Here, although the screen information is generated by the medical service support device 20 as an example of the embodiment, this is merely an example, and the screen information may be generated by another device such as the endoscope part management device 12 or the like, for example.

As described in detail later, the medical service support screen 114 includes a graph, a comment, and the like that visualize the difference degree between the operation rate of each information processing apparatus 36 for each examination time slot on the peak operation date and the information processing apparatus operation rate reference value. The difference degree between the operation rate of the information processing apparatus 36 and the information processing apparatus operation rate reference value indicates a degree to which the operation rate of the information processing apparatus 36 deviates from the information processing apparatus operation rate reference value, and corresponds to, for example, a value obtained by subtracting the operation rate of the information processing apparatus 36 from the information processing apparatus operation rate reference value, or to the ratio of the operation rate of the information processing apparatus 36 to the information processing apparatus operation rate reference value.

Further, the medical service support information includes, as the screen information, information processing apparatus related information related to the information processing apparatus 36 of which the operation rate is within an operation rate range defined based on the information processing apparatus operation rate reference value. The information processing apparatus related information is an example of the “medical device related information” according to the present disclosed technology. Further, in the present first embodiment, as an example of the operation rate range defined based on the information processing apparatus operation rate reference value, the operation rate range equal to or greater than the information processing apparatus operation rate reference value is applied. Further, in the present first embodiment, the information processing apparatus ID is applied as an example of the information processing apparatus related information. As described in detail later, on the medical service support screen 114, the information processing apparatus 36 of which the operation rate is within the operation rate range equal to or greater than the information processing apparatus operation rate reference value is visualized and displayed so as to be specifiable.

Further, in the information processing apparatus related information, information processing apparatus quantity specification information (an example of medical device quantity specification information) that is capable of specifying a quantity of the information processing apparatuses 36 of which the operation rate is within the operation rate range equal to or greater than the information processing apparatus operation rate reference value is included. As described in detail later, on the medical service support screen 114, the quantity of the information processing apparatuses 36 of which the operation rate is within the operation rate range equal to or greater than the information processing apparatus operation rate reference value is visualized and displayed so as to be specifiable.

In step ST216, the transmission unit 98B transmits the medical service support information, which is generated in step ST214, to the endoscope part management device main body 68 via the communication I/F 104 (see FIG. 7). After the process in step ST216 is executed, the medical service support process proceeds to step ST218.

In step ST218, the transmission unit 98B determines whether or not a condition for ending the medical service support process (hereinafter, referred to as a “medical service support process end condition”) is satisfied. Examples of the medical service support process end condition include a condition that an instruction for ending the medical service support process is received by the reception device (not shown).

In step ST218, in a case where the medical service support process end condition is not satisfied, a negative determination is made, and the medical service support process proceeds to step ST200. In step ST218, in a case where the medical service support process end condition is satisfied, a positive determination is made, and the medical service support process is ended.

In a case where the medical service support information is transmitted by executing the process of step ST216, as described above, the medical service support information is received through the support device communication I/F 84 of the endoscope part management device 12 (see step ST110 shown in FIG. 10). As shown in step ST112 shown in FIG. 10, the display control unit 78D of the endoscope part management device 12 generates the medical service support screen 114 based on the medical service support information, and the generated medical service support screen 114 is displayed on the display 72 (FIGS. 1 and 3) (see FIG. 12).

In the following, in the present first embodiment, for convenience of explanation, a case will be described where the operation status of each information processing apparatus 36, which is installed in each of four examination rooms 30, is presented to the user or the like via the display 72 as the medical service support screen 114. Further, the four examination rooms 30 are a first examination room, a second examination room, a third examination room, and a fourth examination room. Further, in the following, for convenience of explanation, the information processing apparatus 36 installed in the first examination room is called a first information processing apparatus, the information processing apparatus 36 installed in the second examination room is called a second information processing apparatus, the information processing apparatus 36 installed in the third examination room is called a third information processing apparatus, and the information processing apparatus 36 installed in the fourth examination room is called a fourth information processing apparatus.

As an example, as shown in FIG. 12, on the medical service support screen 114, a screen title display field 114A, an operation rate calculation target date display field 114B, an excess quantity display field 114C, an explanatory note display field 114D, an examination support graph 114E, an examination support comment display field 114F, and a guidance comment display field 114G are displayed.

In the screen title display field 114A, a title that can be recognized as a screen showing the operation status of the information processing apparatus 36 is displayed. In the example shown in FIG. 12, an “operation status of the information processing apparatus” is displayed in the screen title display field 114A. A period during which the information processing apparatus 36 is being operated can be considered as a period during which the examination room 30 is used for endoscopy, and therefore, in the screen title display field 114A, for example, a title such as “a use status of the examination room” or “an operation status of the information processing apparatus (a use status of examination room)” may be displayed.

In the operation rate calculation target date display field 114B, a date, which is a calculation target of the operation rate in step ST212 included in the medical service support process shown in FIG. 11, that is, the peak operation date derived in step ST206 included in the medical service support process shown in FIG. 11, is displayed. In the example shown in FIG. 12, the peak operation date is represented by the date and day.

In the excess quantity display field 114C, the quantity of the information processing apparatuses 36 of which the operation rate exceeds the upper limit guideline is displayed. In the example shown in FIG. 12, in the excess quantity display field 114C, a comment such as “Quantity exceeding upper limit guideline: ¼” is displayed as the recognizable information that one information processing apparatus 36 out of the four information processing apparatuses 36 exceeds the upper limit guideline. Here, the upper limit guideline refers to the information processing apparatus operation rate reference value calculated by executing the process of step ST210 included in the medical service support process shown in FIG. 11. The upper limit guideline is not limited to this, and may be, for example, a value obtained by finely adjusting the information processing apparatus operation rate reference value.

In the explanatory note display field 114D, an explanatory note, which is related to the examination support graph 114E, is displayed. In the example shown in FIG. 12, in the explanatory note display field 114D, an explanatory note of the first information processing apparatus installed in the first examination room, an explanatory note of the second information processing apparatus installed in the second examination room, an explanatory note of the third information processing apparatus installed in the third examination room, and an explanatory note of the fourth information processing apparatus installed in the fourth examination room are displayed. Further, in the example shown in FIG. 12, in the explanatory note display field 114D, an upward arrow, which indicates that the operation rate of the second information processing apparatus exceeds the upper limit guideline, is also displayed.

The examination support graph 114E is an example of a “figure” according to the present disclosed technology. The examination support graph 114E is a graph showing a time change of the operation rate calculated by executing the process of step ST212 included in the medical service support process shown in FIG. 11. A horizontal axis of the examination support graph 114E is the examination time slot of the peak operation date, and a vertical axis of the examination support graph 114E is the operation rate of the information processing apparatus 36.

In the examination support graph 114E, it is shown that the operation rate of the second information processing apparatus in the endoscopy around 11:00 exceeds the information processing apparatus operation rate reference value as the upper limit guideline, and in other time slots, none of the operation rates of the first to fourth information processing apparatuses reach the information processing apparatus operation rate reference value. Further, in the examination support graph 114E, it is shown that the difference degree between each of the operation rates of the first to fourth information processing apparatuses and the information processing apparatus operation rate reference value is visualized for each examination time slot.

Further, in the examination support graph 114E, the operation rate range exceeding the upper limit guideline of the operation rate, that is, the operation rate range equal to or greater than the information processing apparatus operation rate reference value, is displayed in a manner distinguishable from other regions. Further, in the examination support graph 114E, the range indicating a peak time slot is displayed in a manner distinguishable from other regions. The peak time slot refers to an examination time slot in which the average value of the operation rates of the information processing apparatus 36 is the highest among the examination time slots. In the example shown in FIG. 12, around 11:00 is displayed as the peak time slot in a manner distinguishable from other regions (in the example shown in FIG. 12, a broken line frame).

In the examination support comment display field 114F, a comment contributing to performing an endoscopy is displayed. Specifically, a comment which indicates a correlation between the quantity of the information processing apparatuses 36 of which the operation rate is within the operation rate range equal to or greater than the information processing apparatus operation rate reference value as the upper limit guideline and the number of examinations (for example, the number of times an endoscopy can be performed) is displayed in the examination support comment display field 114F. In the example shown in FIG. 12, in the examination support comment display field 114F, a comment such as “The number of examinations may be increased because the number of information processing apparatuses of which the operation rate exceeds the upper limit guideline is half or less” is displayed. The comment, which is displayed in the examination support comment display field 114F, is an example of a “comment” according to the present disclosed technology.

In the guidance comment display field 114G, a guidance comment is displayed. In the example shown in FIG. 12, in the guidance comment display field 114G, a comment is displayed indicating that the operation status of the endoscope 24 can be checked on the next page of the medical service support screen 114. Here, although a comment which indicates that the operation status of the endoscope 24 can be checked on the next page of the medical service support screen 114 is shown as an example, the present disclosure is not limited to this, and it may be a comment indicating a page of the medical service support screen 114 and a medical device where the operation status can be checked.

As described above, in the medical service support device 20 according to the present first embodiment, the information processing apparatus operation rate reference value is derived based on the standard operation time during which the information processing apparatus 36 used in the endoscope handling service is being operated and on the examination standby time. In the medical service support device 20, the medical service support information, which is obtained in accordance with the difference degree between the operation rate of the information processing apparatus 36 and the information processing apparatus operation rate reference value, is transmitted to the endoscope part management device 12. In the endoscope part management device 12, the medical service support screen 114 is generated based on the medical service support information and is displayed on the display 72. Since the medical service support screen 114 is presented to the user or the like in a state in which the medical service support information is visualized, the user or the like ascertains the difference degree between the operation rate of the information processing apparatus 36 and the information processing apparatus operation rate reference value. Therefore, according to the present configuration, it is possible to support the efficient performance of the endoscope handling service.

Further, the medical service support information includes information related to the information processing apparatus 36 of which the operation rate is within the operation rate range equal to or greater than the information processing apparatus operation rate reference value. In the endoscope part management device 12, the medical service support screen 114 is generated based on the medical service support information and is displayed on the display 72. On the medical service support screen 114, since the information (for example, the examination support graph 114E) which is related to the information processing apparatus 36 of which the operation rate is within the operation rate range equal to or greater than the information processing apparatus operation rate reference value is presented to the user in a visualized state, the user or the like ascertains the information that is related to the information processing apparatus 36 of which the operation rate is within the operation rate range equal to or greater than the information processing apparatus operation rate reference value. Therefore, according to the present configuration, the user or the like can accurately ascertain the information related to the information processing apparatus 36 of which the operation rate is within the operation rate range equal to or greater than the information processing apparatus operation rate reference value, as compared with the case where the medical service support information includes only the operation rate of the information processing apparatus 36.

Further, the medical service support information includes information related to the information processing apparatus 36 of which the operation rate is within the operation rate range equal to or greater than the information processing apparatus operation rate reference value on the peak operation date. In the endoscope part management device 12, the medical service support screen 114 is generated based on the medical service support information and is displayed on the display 72. On the medical service support screen 114, since the information (for example, the examination support graph 114E) which is related to the information processing apparatus 36 of which the operation rate is within the operation rate range equal to or greater than the information processing apparatus operation rate reference value on the peak operation date is presented to the user in a visualized state, the user or the like ascertains the information that is related to the information processing apparatus 36 of which the operation rate is within the operation rate range equal to or greater than the information processing apparatus operation rate reference value on the peak operation date. Therefore, according to the present configuration, the user or the like can accurately ascertain the information related to the information processing apparatus 36 of which the operation rate is within the operation rate range equal to or greater than the information processing apparatus operation rate reference value on the peak operation date, as compared with the case where the medical service support information includes only the operation rate of the information processing apparatus 36.

Further, the peak operation date is a date on which an endoscopy is performed the highest number of times. Therefore, according to the present configuration, the user or the like can accurately ascertain the information related to the information processing apparatus 36 of which the operation rate is within the operation rate range equal to or greater than the information processing apparatus operation rate reference value on the date on which an endoscopy is performed the highest number of times, as compared with the case where the medical service support information includes only the operation rate of the information processing apparatus 36.

Further, in the medical service support information, as information related to the information processing apparatus 36, information processing apparatus quantity specification information that is capable of specifying a quantity of the information processing apparatuses 36 of which the operation rate is within the operation rate range equal to or greater than the information processing apparatus operation rate reference value is included. On the medical service support screen 114, the quantity of the information processing apparatuses 36 of which the operation rate is within the operation rate range equal to or greater than the information processing apparatus operation rate reference value is visualized and displayed so as to be specifiable. Therefore, according to the present configuration, the user or the like can ascertain the quantity of the information processing apparatuses 36 of which the operation rate is within the operation rate range equal to or greater than the information processing apparatus operation rate reference value.

Further, the standard operation time, during which the information processing apparatus 36 used in the endoscope handling service is being operated, is time based on the operation time of all the information processing apparatuses 36 on the peak operation date. In the medical service support device 20, the information processing apparatus operation rate reference value is derived based on the standard operation time during which the information processing apparatus 36 is being operated and on the examination standby time. Therefore, according to the present configuration, a value with high reliability can be obtained as the information processing apparatus operation rate reference value on the peak operation date, as compared with a case where the standard operation time, during which the information processing apparatus 36 used in the endoscope handling service is being operated, is defined as time based on the operation time of all the information processing apparatuses 36 during time periods other than the peak operation date.

Further, the information processing apparatus operation rate reference value is a value based on a ratio of the information processing apparatus standard operation time with respect to the sum of the shortest examination standby time, which is defined as the shortest time of the examination standby time, and the information processing apparatus standard operation time. Therefore, according to the present configuration, it is possible to increase the information processing apparatus operation rate reference value, which is the target for comparison of the operation rate of the information processing apparatus 36, as compared with the case where the ratio of the information processing apparatus standard operation time with respect to the sum of the time longer than the shortest examination standby time and the information processing apparatus standard operation time is used as the information processing apparatus operation rate reference value.

Further, the information processing apparatus operation rate reference value is a value obtained by multiplying the ratio of the information processing apparatus standard operation time with respect to the sum of the shortest examination standby time, which is defined as the shortest time of the examination standby time, and the information processing apparatus standard operation time, by the adjustment factor. For example, the adjustment factor is defined according to an external factor (for example, rest time for endoscopy workers and/or potential problems) that affects at least one of the shortest examination standby time or the information processing apparatus standard operation time. Therefore, according to the present configuration, an appropriate value can be obtained as the information processing apparatus operation rate reference value, which is the target for comparison of the operation rate of the information processing apparatus 36, even in a case where there is an external factor that affects at least one of the shortest examination standby time or the information processing apparatus standard operation time, as compared with the case where only the ratio of the information processing apparatus standard operation time with respect to the sum of the shortest examination standby time and the information processing apparatus standard operation time is used as the information processing apparatus operation rate reference value.

Further, the operation rate of the information processing apparatus 36 is calculated based on the examination standby time (see FIG. 2) and on the operation time of the information processing apparatus 36. The operation time of the information processing apparatus 36 is time from when an endoscopy is started to when the endoscopy is ended. That is, the operation rate of the information processing apparatus 36 represents a tight status degree in the performance of the endoscopy. Therefore, according to the present configuration, the user or the like can estimate the tight status in the performance of the endoscopy by referring to the operation rate of the information processing apparatus 36.

Further, the standard operation time, during which the information processing apparatus 36 used in the endoscope handling service is being operated, is an average value of the operation time of all the information processing apparatuses 36 on the peak operation date. Therefore, according to the present configuration, an appropriate information processing apparatus operation rate reference value can be calculated for comparison with each operation rate of all the information processing apparatuses 36, as compared with the case of calculating the standard operation time by using only the operation time of one information processing apparatus 36 on the peak operation date.

Further, the medical service support information is information that is capable of being represented via a comment and a figure (for example, the examination support graph 114E) on the display 72. Therefore, according to the present configuration, the user or the like can visually ascertain the medical service support information.

In the present first embodiment described above, although an example of the embodiment has been described in which the information processing apparatus quantity specification information (hereinafter, also referred to as “first quantity specification information”), which can specify the quantity of the information processing apparatuses 36 of which the operation rate is within the operation rate range equal to or greater than the information processing apparatus operation rate reference value, is included in the medical service support information, the present disclosed technology is not limited to this. For example, the medical service support information may include notification information for notifying that the operation status of the information processing apparatus 36 is tight, together with the first quantity specification information, or instead of the first quantity specification information.

For example, the notification information is information that includes a tight time slot, which is a time slot where the operation status of the information processing apparatus 36 is tight (here, for example, the examination time slot), and second quantity specification information. The second quantity specification information refers to information that is capable of specifying the quantity of the information processing apparatuses 36 operating during the tight time slot. In a case where the medical service support information includes the notification information, the tight time slot and the second quantity specification information are presented by the presentation device in a comparable state.

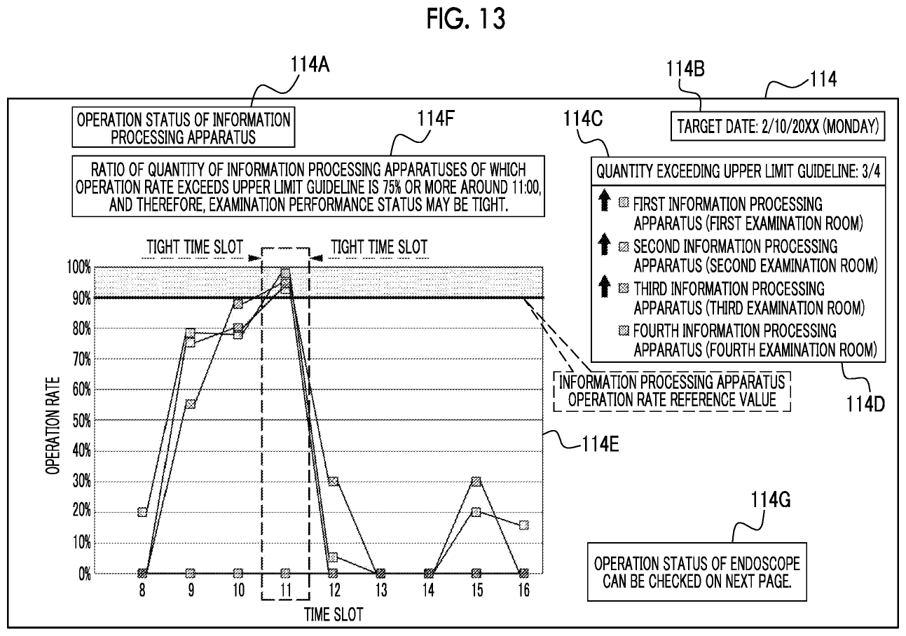

For example, as shown in FIG. 13, on the examination support graph 114E of the medical service support screen 114, the tight time slot and the quantity specified based on the second quantity specification information, that is, the quantity of the information processing apparatuses 36 operating during the tight time slot, are displayed in a comparable state. The tight time slot is displayed in a manner distinguishable from other regions in the examination support graph 114E. Further, on the examination support graph 114E, three marks, which indicate the first to third information processing apparatuses having operation rates exceeding the information processing apparatus operation rate reference value, are plotted in the tight time slot.

Further, in the excess quantity display field 114C of the medical service support screen 114 shown in FIG. 13, a comment is displayed indicating that the quantity of the information processing apparatuses 36 of which the operation rate exceeds the information processing apparatus operation rate reference value as the upper limit guideline is three out of four. Further, in the examination support comment display field 114F of the medical service support screen 114 shown in FIG. 13, a comment is displayed indicating the tight status of the operation status of the information processing apparatus 36. Specifically, a comment indicating the tight time slot and the tight status degree of the endoscopy is displayed in the examination support comment display field 114F. In the example shown in FIG. 13, in the examination support comment display field 114F, a comment such as “The ratio of the quantity of the information processing apparatuses of which the operation rate exceeds the upper limit guideline is 75% or more around 11:00, and therefore, the examination performance status may be tight” is displayed.

In this way, by including the notification information for notifying that the operation status of the information processing apparatus 36 is tight in the medical service support information, it is possible for the user or the like to recognize that the operation status of the information processing apparatus 36 is tight. Further, the notification information is information that includes the tight time slot and the second quantity specification information, and the tight time slot and the second quantity specification information are presented by the presentation device in a comparable state, and therefore, it is possible for the user or the like to recognize the relationship between the tight time slot and the quantity of the information processing apparatuses 36 operating during the tight time slot.

In the first embodiment described above, although an example of the embodiment has been described in which the information processing apparatus operation rate reference value is derived regardless of the type of endoscopy, and the medical service support information is output regardless of the type of endoscopy, the present disclosed technology is not limited to this. For example, the CPU 98 of the support device server 74 may derive the information processing apparatus operation rate reference value for each type of endoscopy and output the medical service support information for each type of endoscopy.

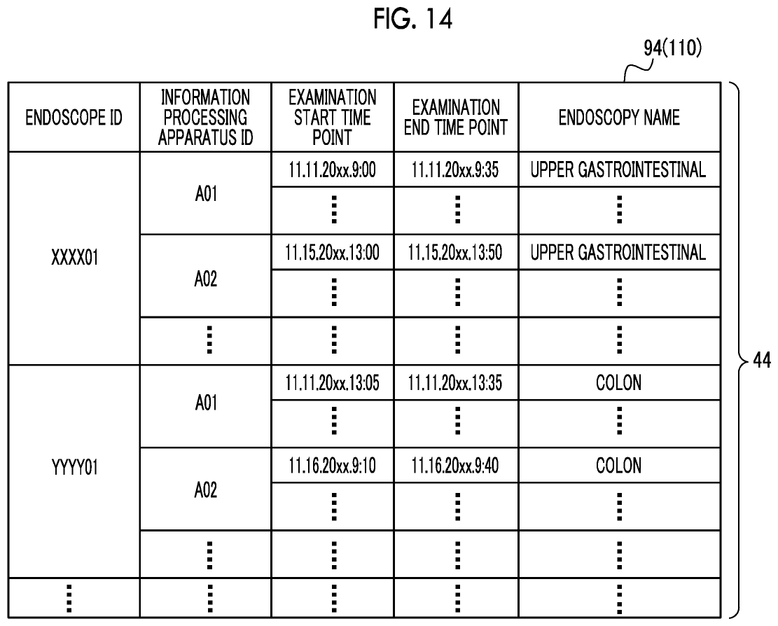

In order to realize this, as an example, as shown in FIG. 14, the endoscopy information 44 stored in the endoscopy management databases 94 and 110 further includes an endoscopy name. Specifically, the endoscopy name is associated with the examination start time point and the examination end time point. Similarly, in the washer management databases 96 and 112, the endoscopy name is associated with the examination start time point and the examination end time point.

Each of the processes of step ST206 to step ST216 of the medical service support process shown in FIG. 11 is performed for each endoscopy name based on the endoscopy management database 110 shown in FIG. 14. Further, on the display 72, the medical service support screen 114 (see FIGS. 12 and 13) is displayed in a manner distinguishable for each endoscopy name. For example, the endoscopy name is displayed in the medical service support screen 114. Accordingly, it is possible to specify which type of endoscopy the medical service support screen 114 displayed on the display 72 is.

As described above, in the present example, the medical service support information is transmitted from the medical service support device 20 to the endoscope part management device 12 for each type of endoscopy, and the medical service support screen 114, which is based on the medical service support information, is displayed on the display 72 for each type of endoscopy. Therefore, according to the present example, it is possible to support the efficient performance of the endoscope handling service for each type of endoscopy.

In the first embodiment described above, although an example of the embodiment has been described in which the date on which an endoscopy is performed the highest number of times is defined as the peak operation date, the present disclosed technology is not limited to this. For example, as the peak operation date, a date on which the number of times the examination standby time falls below a threshold value (for example, 3 minutes) is equal to or greater than a reference number of times may be set as the peak operation date. In this case, it is possible to specify the peak operation date even in a case where the number of times an endoscopy is performed cannot be ascertained. In other words, the operation degree of the medical device may be indicated by a value based on the number of times the examination standby time falls below the threshold value, and the first reference degree may be indicated by the reference number of times. The number of times the standby time falls below the threshold value indicates the number of times the standby time for each predetermined period, such as for each day or for each hour, falls below the threshold value. Further, in a case where there are a plurality of medical devices that can be acquired by the medical service support device, the number of times the standby time falls below the threshold value indicates the total number of times the standby time falls below the threshold value in the plurality of medical devices. The threshold value may correspond to a preparation period required from the endoscopy end to the next endoscopy start in a medical installation facility, may be a fixed value, or may be a variable value set or changed by the user or the like. Further, the threshold value may be a variation value calculated based on the standby time of the endoscope procedure service performed in the past. For example, the average value of the standby time of the endoscope procedure service performed in the past, the minimum value of the standby time of the endoscope procedure service performed in the past, or a value obtained by adding or multiplying a predetermined value to the minimum value may be used. Further, the reference number of times corresponding to the first reference degree is a default value. More specifically, it is a default value for determining a period during which endoscopy is intensively performed, such as the peak date or the peak time. The default value may be a fixed value set in advance, or a variable value changed according to a user input. Further, the default value may be a variation value calculated based on the number of times the standby time in the past falls below the threshold value. For example, the default value may be an average value of the number of times the standby time in the past falls below the threshold value or a variation value calculated based on the number of times the standby time in the past falls below the threshold value, for example, the maximum value of the number of times the standby time in the past falls below the threshold value or a value obtained by adding or multiplying a predetermined value to the maximum value. Further, in a case where the operation degree of the medical device indicates the number of times the standby time falls below the threshold value for each first period (for example, one day), the highest value, which is within the number of times the standby time falls below the threshold value for each first period in a second period (for example, one week) longer than the first period, may be set as a defined value.

In the first embodiment described above, although the peak operation date is exemplified, the present disclosed technology is not limited to this, and the date on which the number of times an endoscopy is performed exceeded the default number of times may be applied. The default number of times (a default value) may be a fixed value or a variable value that is changed according to an instruction received by the reception device 70 or the like.

In the first embodiment described above, although the peak operation date is exemplified, the present disclosed technology is not limited to this, and the peak operation time period may be specified in a unit of time other than day, such as peak operation month or peak operation year.

In the first embodiment described above, although a broken line graph is exemplified as the examination support graph 114E, the present disclosed technology is not limited to this, and other graphs such as a bar graph, a pie chart, and a band graph may be used, and figures other than graphs such as a bubble chart may be used.

In the first embodiment described above, although the case has been described where the medical service support information is the examination support graph 114E and the information in which the comment can be represented, the present disclosed technology is not limited to this, and the medical service support information may be information that can be presented in a table. That is, the medical service support information may be any information that can be represented by at least one of a figure, a comment, or a table.

In the first embodiment described above, although the operation rate range that is equal to or greater than the information processing apparatus operation rate reference value is exemplified, the present disclosed technology is not limited to this, and, for example, an operation rate range that is equal to or less than the information processing apparatus operation rate reference value may be applied, and any operation rate range defined based on the information processing apparatus operation rate reference value may be used. In this case as well, similarly to the first embodiment, the relationship between the operation rate range, which is defined based on the information processing apparatus operation rate reference value, and the operation rate of the information processing apparatus 36 may be visualized by using at least one of a graph, a comment, or a table and presented to the user or the like via the display 72.

In the first embodiment described above, although the information processing apparatus operation rate reference value is exemplified as an upper limit guideline, the present disclosed technology is not limited to this. For example, the information processing apparatus operation rate reference value may be an average operation rate of a plurality of information processing apparatuses 36 (for example, all the information processing apparatuses 36) in a period designated by the user or the like or may be a median value of the operation rates. Accordingly, it is possible for the user or the like to ascertain the correlation between the median value of the average operation rate or the operation rate, and the operation rate of each information processing apparatus 36. The information processing apparatus operation rate reference value is not limited to the average value and the median value and may be other statistical values such as the maximum value, the minimum value, or the most frequent value.

In the first embodiment described above, although an example of the embodiment has been described in which a comment such as “The number of examinations may be increased because the number of information processing apparatuses of which the operation rate exceeds the upper limit guideline is half or less” is displayed in the examination support comment display field 114F, the present disclosed technology is not limited to this. For example, in a case where the operation rate of all the information processing apparatuses 36 exceeds the upper limit guideline, a message (a message expressing that the operation status of the information processing apparatus 36 is tight) such as “The information processing apparatus has no operating capacity” may be displayed in the examination support comment display field 114F, and in a case where the operation rate of at least one information processing apparatus 36 is equal to or less than the upper limit guideline, a message (a message expressing that the operation of the information processing apparatus 36 is not tight) such as “The information processing apparatus has operating capacity” may be displayed in the examination support comment display field 114F.

Further, in the medical service support screen 114, a symbol mark for each of the first to fourth information processing apparatuses and marks such as “A”, “B”, and “C” that are selectively assigned to each symbol mark may be displayed. The “A” mark is a mark indicating that the information processing apparatus 36 has operating capacity, the “B” mark is a mark indicating that the information processing apparatus 36 has a small amount of operating capacity, and the “C” mark is a mark indicating that the information processing apparatus 36 has no operating capacity. Further, a tightness degree may be visualized by changing a display aspect such as a color and/or a font of each of the symbol marks of the first to fourth information processing apparatuses to highlight them.

In the first embodiment described above, although an example of the embodiment has been described in which the operation of the information processing apparatus 36 is started by turning on the examination start button 38A, and the operation of the information processing apparatus 36 is stopped by turning off the examination start button 38A, the present disclosed technology is not limited to this. The operation of the information processing apparatus 36 may be started on a condition that the barcode 42 is read by the barcode reader 40, and the operation of the information processing apparatus 36 may be stopped on a condition that the barcode 42 is read again by the barcode reader 40. The same applies to the start and stop of the operation of the washer 52.

An example has been described in which the start operation and the stop operation of the information processing apparatus 36 are executed by pressing down the examination start button 38A, but this is merely an example. The examination start button 38A is not limited to a physical button. The operation of the information processing apparatus 36 may be started or the operation of the information processing apparatus 36 may be stopped by detecting nearness of at least one softkey displayed on a touch panel, such as a touch panel display. Further, the start operation and the stop operation of the information processing apparatus 36 may be controlled by a voice input and/or a gesture input. Further, a noncontact human sensor may be provided in a default region of the examination room 30 or the information processing apparatus 36, and the operation of the information processing apparatus 36 may be started or the operation of the information processing apparatus 36 may be stopped by causing the noncontact human sensor to detect a human body. The same applies to the control of the start operation and the stop operation by using the washing start button 60A of the washer 52.

### Second Embodiment

In the first embodiment described above, although an example of the embodiment has been described in which the performance of the endoscope handling service is supported by using the medical service support information obtained based on the operation rate of the information processing apparatus 36, which is an example of the “medical device” according to the present disclosed technology, in the second embodiment, a case will be described where the performance of the endoscope handling service is supported by using the medical service support information obtained based on the operation rate of the endoscope 24. Further, in the present second embodiment, the same components as those in the first embodiment are designated by the same reference numerals, the description thereof will be omitted, and the parts different from those in the first embodiment will be described.

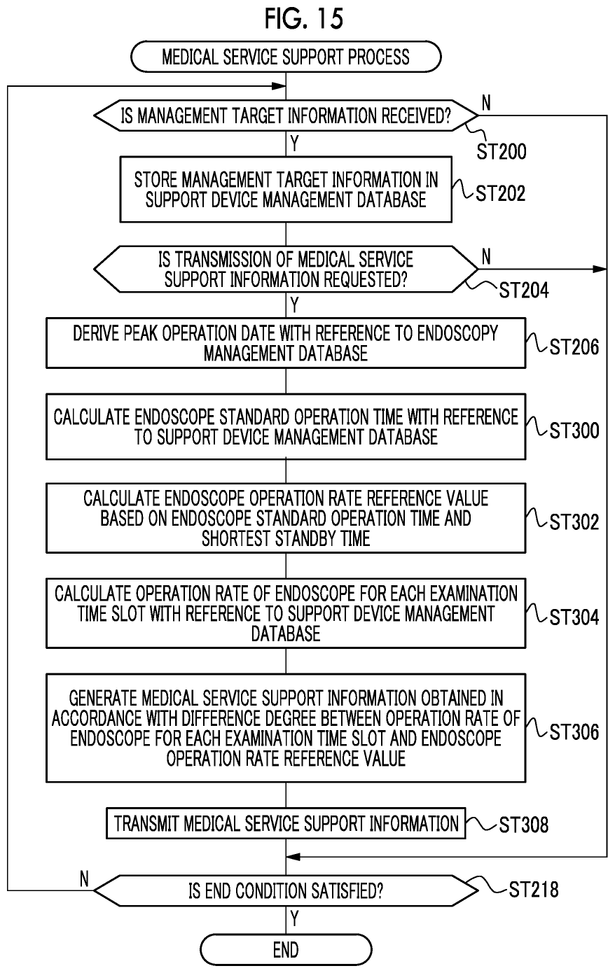

FIG. 15 shows an example of a flow of a medical service support process according to the present second embodiment.

The flowchart shown in FIG. 15 is different from the flowchart shown in FIG. 11 in that it includes step ST300 to step ST308 instead of step ST208 to step ST216. Therefore, in the following description, steps in the flowchart shown in FIG. 11 that are the same as those in the flowchart shown in FIG. 15 are given the same step numbers, the description thereof will be omitted, and steps different from those included in the flowchart shown in FIG. 11 will be described.

In the medical service support device shown in FIG. 15, in step ST300, the derivation unit 98E calculates endoscope standard operation time for the peak operation date derived in step ST206 with reference to the support device management database 113. After the process in step ST300 is executed, the medical service support process proceeds to step ST302.

In step ST300, the endoscope standard operation time is a statistical value of time based on the operation time of the endoscope 24 on the peak operation date, that is, the operation time of all endoscopes 24 (endoscope operation time for all the endoscopes 24) on the peak operation date. Here, the endoscope operation time refers to the operation time of the endoscope 24. The endoscope operation time is an example of the “operation time of the endoscope” according to the present disclosed technology and is time from when the examination start button 38A is turned on in step ST16 shown in FIG. 2 to when the washing of the endoscope 24 by the washer 52 is ended in step ST32 shown in FIG. 2.

For example, the endoscope standard operation time is an average value of the operation time of all the endoscopes 24 on the peak operation date. The operation time of the endoscope 24 is specified based on the examination start time point of the endoscopy information 44 stored in the endoscopy management database 110 and on the washing end time point of the washing information 66 stored in the washer management database 112.

Here, although the average value of the operation time of all the endoscopes 24 is exemplified as the endoscope standard operation time, this is merely an example, and it may be a statistical value such as a median value, the most frequent value, the maximum value, the minimum value, or a percentile of the operation time of all endoscopes 24. Further, the endoscope standard operation time may be calculated within a time range (for example, within a range of substantially 5 minutes to 120 minutes) predetermined with an upper limit value and a lower limit value.

In step ST302, the derivation unit 98E calculates an endoscope operation rate reference value based on the endoscope standard operation time calculated in step ST300 and on the shortest endoscope standby time. After the process in step ST302 is executed, the medical service support process proceeds to step ST304. In the present step ST302, the shortest endoscope standby time refers to the shortest standby time until the endoscope 24 which is washed by the washer 52 is used in the endoscopy. In the medical service support process according to the present second embodiment, the shortest endoscope standby time refers to the shortest time of the standby time from when the washing of the endoscope 24 by the washer 52 is ended to when an endoscopy is started. Here, as an example of the shortest endoscope standby time, predetermined time (for example, pre-designated time between 1 minute and 3 minutes) is applied as the shortest time from when the washing of the endoscope 24 by the washer 52 is ended (for example, from when the process of step ST32 shown in FIG. 2 is ended) to when the examination start button 38A is turned on (for example, to when the process of step ST16 shown in FIG. 2 is performed) in a case where an endoscopy is performed using the washed endoscope 24. The shortest endoscope standby time may be a fixed value or a variable value that is changed according to an instruction received by the reception device 70 or the like.

In step ST302, the endoscope operation rate reference value is a value based on a ratio of the endoscope standard operation time with respect to time including the endoscope standard operation time calculated in step ST300 and the shortest endoscope standby time. Examples of the endoscope operation rate reference value include a value obtained by multiplying the ratio of the endoscope standard operation time with respect to the sum of the endoscope standard operation time calculated in step ST300 and the shortest endoscope standby time, by an adjustment factor. Examples of the adjustment factor according to the present second embodiment include 0.9. The adjustment factor of “0.9” is merely an example, and the adjustment factor may be another decimal number less than 1. Further, the adjustment factor may be a fixed value or a variable value.

Further, although an example of the embodiment has been described in which the ratio of the endoscope standard operation time with respect to the sum of the endoscope standard operation time and the shortest endoscope standby time is multiplied by the adjustment factor, this is merely an example, and a value which is obtained by adding or subtracting an adjustment value to the ratio of the endoscope standard operation time with respect to the sum of the endoscope standard operation time and the shortest endoscope standby time may be used as the endoscope operation rate reference value.

In step ST304, the derivation unit 98E derives an operation rate of each endoscope 24 for each examination time slot on the peak operation date derived in step ST206 with reference to the support device management database 113. After the process in step ST304 is executed, the medical service support process proceeds to step ST306.

The operation rate of the endoscope 24 is calculated by the derivation unit 98E according to the expression “(operation time of endoscope 24)/{(operation time of endoscope 24)+(endoscope standby time)}” for each examination time slot on the peak operation date. In this expression, the endoscope standby time refers to time from when washing of the endoscope 24 by the washer 52 is ended to when an endoscopy is started. The endoscope standby time is an example of “standby time” according to the present disclosed technology.

In step ST306, the generation unit 98F generates the medical service support information obtained in accordance with a difference degree between the operation rate of each endoscope 24 for each examination time slot on the peak operation date (the operation rate calculated in step ST304) and the endoscope operation rate reference value calculated in step ST302. After the process in step ST306 is executed, the medical service support process proceeds to step ST308.

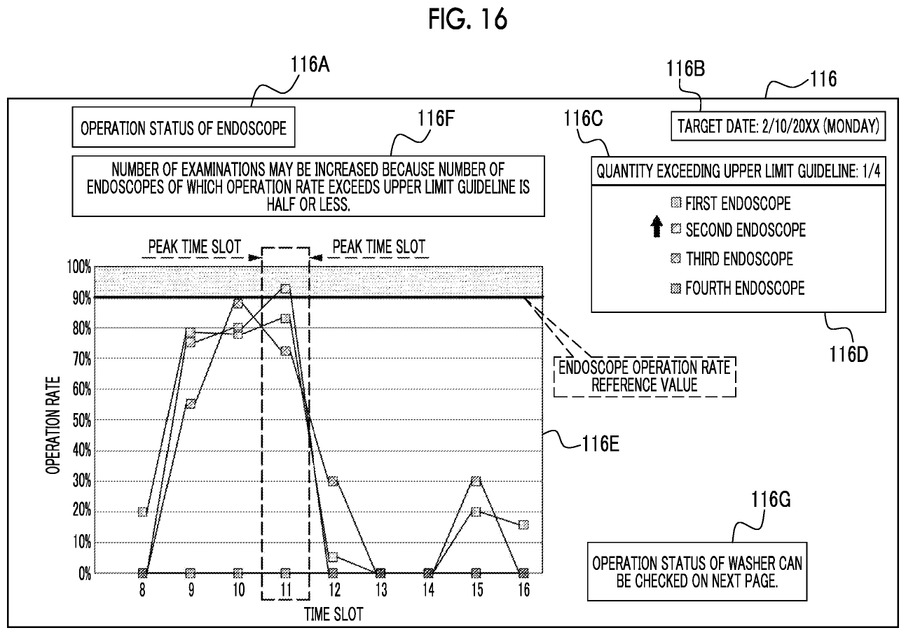

The medical service support information is information that is capable of being represented via a comment, a figure, or the like by a presentation device (here, the display 72 as an example) and includes screen information indicating a medical service support screen 116 (see FIG. 16). As described in detail later, the medical service support screen 116 includes a graph, a comment, and the like that visualize the difference degree between the operation rate of each endoscope 24 for each examination time slot on the peak operation date and the endoscope operation rate reference value. The difference degree between the operation rate of the endoscope 24 and the endoscope operation rate reference value indicates a degree to which the operation rate of the endoscope 24 deviates from the endoscope operation rate reference value, and corresponds to, for example, a value obtained by subtracting the operation rate of the endoscope 24 from the endoscope operation rate reference value, or to the ratio of the operation rate of the endoscope 24 to the endoscope operation rate reference value.

Further, the medical service support information includes, as the screen information, endoscope related information related to the endoscope 24 of which the operation rate is within an operation rate range defined based on the endoscope operation rate reference value. The endoscope related information is an example of the “medical device related information” according to the present disclosed technology. Further, in the present second embodiment, as an example of the operation rate range defined based on the endoscope operation rate reference value, the operation rate range equal to or greater than the endoscope operation rate reference value is applied. Further, in the present second embodiment, the endoscope ID is applied as an example of the endoscope related information. As described in detail later, on the medical service support screen 116, the endoscope 24 of which the operation rate is within the operation rate range equal to or greater than the endoscope operation rate reference value is visualized and displayed so as to be specifiable.

Further, in the endoscope related information, endoscope quantity specification information (an example of the medical device quantity specification information) that is capable of specifying a quantity of the endoscopes 24 of which the operation rate is within the operation rate range equal to or greater than the endoscope operation rate reference value is included. As described in detail later, on the medical service support screen 116, the quantity of the endoscopes 24 of which the operation rate is within the operation rate range equal to or greater than the endoscope operation rate reference value is visualized and displayed so as to be specifiable.

In step ST308, the transmission unit 98B transmits the medical service support information, which is generated in step ST306, to the endoscope part management device main body 68 via the communication I/F 104 (see FIG. 7). After the process in step ST308 is executed, the medical service support process proceeds to step ST218.

In a case where the medical service support information is transmitted by executing the process of step ST308, as described above, the medical service support information is received through the support device communication I/F 84 of the endoscope part management device 12 (see step ST110 shown in FIG. 10). As shown in step ST112 shown in FIG. 10, the display control unit 78D of the endoscope part management device 12 generates the medical service support screen 116 based on the medical service support information, and the generated medical service support screen 116 is displayed on the display 72 (FIGS. 1 and 3) (see FIG. 12).

In the following, in the present second embodiment, for convenience of explanation, a case will be described where operation statuses of a first endoscope, a second endoscope, a third endoscope, and a fourth endoscope are presented, as the four endoscopes 24, to the user or the like via the display 72 as the medical service support screen 116.

As an example, as shown in FIG. 16, on the medical service support screen 116, a screen title display field 116A, an operation rate calculation target date display field 116B, an excess quantity display field 116C, an explanatory note display field 116D, an examination support graph 116E, an examination support comment display field 116F, and a guidance comment display field 116G are displayed.

In the screen title display field 116A, a title that can be recognized as a screen showing the operation status of the endoscope 24 is displayed. In the example shown in FIG. 16, an “operation status of the endoscope” is displayed in the screen title display field 116A.

In the operation rate calculation target date display field 116B, a date, which is a calculation target of the operation rate in step ST304 included in the medical service support process shown in FIG. 15, that is, the peak operation date derived in step ST206 included in the medical service support process shown in FIG. 15, is displayed. In the example shown in FIG. 16, the peak operation date is represented by the date and day.

In the excess quantity display field 116C, the quantity of the endoscopes 24 of which the operation rate exceeds the upper limit guideline is displayed. In the example shown in FIG. 16, in the excess quantity display field 116C, a comment such as “Quantity exceeding upper limit guideline: ¼” is displayed as the recognizable information that one endoscope 24 out of the four endoscopes 24 exceeds the upper limit guideline. Here, the upper limit guideline refers to the endoscope operation rate reference value calculated by executing the process of step ST302 included in the medical service support process shown in FIG. 15. The upper limit guideline is not limited to this, and may be, for example, a value obtained by finely adjusting the endoscope operation rate reference value.

An explanatory note, which is related to the examination support graph 116E, is displayed in the explanatory note display field 116D. In the example shown in FIG. 16, in the explanatory note display field 116D, an explanatory note of the first endoscope, an explanatory note of the second endoscope, an explanatory note of the third endoscope, and an explanatory note of the fourth endoscope are displayed. Further, in the example shown in FIG. 16, in the explanatory note display field 116D, an upward arrow, which indicates that the operation rate of the second endoscope exceeds the upper limit guideline, is also displayed.

The examination support graph 116E is an example of a “figure” according to the present disclosed technology. The examination support graph 116E is a graph showing a time change of the operation rate calculated by executing the process of step ST304 included in the medical service support process shown in FIG. 15. A horizontal axis of the examination support graph 116E is the examination time slot of the peak operation date, and a vertical axis of the examination support graph 116E is the operation rate of the endoscope 24.

In the examination support graph 116E, it is shown that the operation rate of the second endoscope around 11:00 exceeds the endoscope operation rate reference value as the upper limit guideline, and in other time slots, none of the operation rates of the first to fourth endoscopes reach the endoscope operation rate reference value. Further, in the examination support graph 116E, it is shown that the difference degree between each of the operation rates of the first to fourth endoscopes and the endoscope operation rate reference value is visualized for each examination time slot.

Further, in the examination support graph 116E, the operation rate range exceeding the upper limit guideline of the operation rate, that is, the operation rate range equal to or greater than the endoscope operation rate reference value, is displayed in a manner distinguishable from other regions. Further, in the examination support graph 116E, the range indicating a peak time slot is displayed in a manner distinguishable from other regions. The peak time slot refers to an examination time slot in which the average value of the operation rates of the endoscope 24 is the highest among the examination time slots. In the example shown in FIG. 16, around 11:00 is displayed as the peak time slot in a manner distinguishable from other regions (in the example shown in FIG. 16, a broken line frame).

A comment contributing to performing an endoscopy is displayed in the examination support comment display field 116F. Specifically, a comment which indicates a correlation between the quantity of the endoscopes 24 of which the operation rate is within the operation rate range equal to or greater than the endoscope operation rate reference value as the upper limit guideline and the number of examinations (for example, the number of times an endoscopy can be performed) is displayed in the examination support comment display field 116F. In the example shown in FIG. 16, in the examination support comment display field 116F, a comment such as “The number of examinations may be increased because the number of endoscopes of which the operation rate exceeds the upper limit guideline is half or less” is displayed. The comment, which is displayed in the examination support comment display field 116F, is an example of a “comment” according to the present disclosed technology.

A guidance comment is displayed in the guidance comment display field 116G. In the example shown in FIG. 16, in the guidance comment display field 116G, a comment is displayed indicating that the operation status of the washer 52 can be checked on the next page of the medical service support screen 116. Here, although a comment which indicates that the operation status of the washer 52 can be checked on the next page of the medical service support screen 116 is shown as an example, the present disclosure is not limited to this, and it may be a comment indicating a page of the medical service support screen 116 and a medical device where the operation status can be checked.

As described above, in the medical service support device 20 according to the present second embodiment, the endoscope operation rate reference value is derived based on the standard operation time during which the endoscope 24 used in the endoscope handling service is being operated and on the endoscope standby time. In the medical service support device 20, the medical service support information, which is obtained in accordance with the difference degree between the operation rate of the endoscope 24 and the endoscope operation rate reference value, is transmitted to the endoscope part management device 12. In the endoscope part management device 12, the medical service support screen 116 is generated based on the medical service support information and is displayed on the display 72. Since the medical service support screen 116 is presented to the user or the like in a state in which the medical service support information is visualized, the user or the like ascertains the difference degree between the operation rate of the endoscope 24 and the endoscope operation rate reference value. Therefore, according to the present configuration, it is possible to support the efficient performance of the endoscope handling service.

Further, the medical service support information includes information related to the endoscope 24 of which the operation rate is within the operation rate range equal to or greater than the endoscope operation rate reference value. In the endoscope part management device 12, the medical service support screen 116 is generated based on the medical service support information and is displayed on the display 72. On the medical service support screen 116, since the information (for example, the examination support graph 116E) which is related to the endoscope 24 of which the operation rate is within the operation rate range equal to or greater than the endoscope operation rate reference value is presented to the user in a visualized state, the user or the like ascertains the information that is related to the endoscope 24 of which the operation rate is within the operation rate range equal to or greater than the endoscope operation rate reference value. Therefore, according to the present configuration, the user or the like can accurately ascertain the information related to the endoscope 24 of which the operation rate is within the operation rate range equal to or greater than the endoscope operation rate reference value, as compared with the case where the medical service support information includes only the operation rate of the endoscope 24.

Further, the medical service support information includes information related to the endoscope 24 of which the operation rate is within the operation rate range equal to or greater than the endoscope operation rate reference value on the peak operation date. In the endoscope part management device 12, the medical service support screen 116 is generated based on the medical service support information and is displayed on the display 72. On the medical service support screen 116, since the information (for example, the examination support graph 116E) which is related to the endoscope 24 of which the operation rate is within the operation rate range equal to or greater than the endoscope operation rate reference value on the peak operation date is presented to the user in a visualized state, the user or the like ascertains the information that is related to the endoscope 24 of which the operation rate is within the operation rate range equal to or greater than the endoscope operation rate reference value on the peak operation date. Therefore, according to the present configuration, the user or the like can accurately ascertain the information related to the endoscope 24 of which the operation rate is within the operation rate range equal to or greater than the endoscope operation rate reference value on the peak operation date, as compared with the case where the medical service support information includes only the operation rate of the endoscope 24.

Further, the peak operation date is a date on which an endoscopy is performed the highest number of times. Therefore, according to the present configuration, the user or the like can accurately ascertain the information related to the endoscope 24 of which the operation rate is within the operation rate range equal to or greater than the endoscope operation rate reference value on the date on which an endoscopy is performed the highest number of times, as compared with the case where the medical service support information includes only the operation rate of the endoscope 24.

Further, in the medical service support information, endoscope quantity specification information that is capable of specifying a quantity of the endoscopes 24 of which the operation rate is within the operation rate range equal to or greater than the endoscope operation rate reference value is included as the information related to the endoscope 24. On the medical service support screen 116, the quantity of the endoscopes 24 of which the operation rate is within the operation rate range equal to or greater than the endoscope operation rate reference value is visualized and displayed so as to be specifiable. Therefore, according to the present configuration, the user or the like can ascertain the quantity of the endoscopes 24 of which the operation rate is within the operation rate range equal to or greater than the endoscope operation rate reference value.

Further, the standard operation time, during which the endoscope 24 used in the endoscope handling service is being operated, is time based on the operation time of all the endoscopes 24 on the peak operation date. In the medical service support device 20, the endoscope operation rate reference value is derived based on the standard operation time during which the endoscope 24 is being operated and on the endoscope standby time. Therefore, according to the present configuration, a value with high reliability can be obtained as the endoscope operation rate reference value on the peak operation date, as compared with a case where the standard operation time, during which the endoscope 24 used in the endoscope handling service is being operated, is defined as time based on the operation time of all the endoscopes 24 during time periods other than the peak operation date.

Further, the endoscope operation rate reference value is a value based on a ratio of the endoscope standard operation time with respect to the sum of the shortest endoscope standby time defined as the shortest time of the endoscope standby time and the endoscope standard operation time. Therefore, according to the present configuration, it is possible to increase the endoscope operation rate reference value, which is the target for comparison of the operation rate of the endoscope 24, as compared with the case where the ratio of the endoscope standard operation time with respect to the sum of the time longer than the shortest endoscope standby time and the endoscope standard operation time is used as the endoscope operation rate reference value.

Further, the endoscope operation rate reference value is a value obtained by multiplying the ratio of the endoscope standard operation time with respect to the sum of the shortest endoscope standby time, which is defined as the shortest endoscope standby time, and the endoscope standard operation time, by the adjustment factor. For example, the adjustment factor is defined according to an external factor (for example, rest time for endoscopy workers and/or potential problems) that affects at least one of the shortest endoscope standby time or the endoscope standard operation time. Therefore, according to the present configuration, an appropriate value can be obtained as the endoscope operation rate reference value, which is the target for comparison of the operation rate of the endoscope 24, even in a case where there is an external factor that affects at least one of the shortest endoscope standby time or the endoscope standard operation time, as compared with the case where only the ratio of the endoscope standard operation time with respect to the sum of the shortest endoscope standby time and the endoscope standard operation time is used as the endoscope operation rate reference value.

Further, the operation rate of the endoscope 24 is calculated based on the endoscope standby time and on the operation time of the endoscope 24. The operation time of the endoscope 24 is time from when an endoscopy is started to when washing of the endoscope 24 by the washer 52 is ended. That is, the operation rate of the endoscope 24 represents a tight status degree of the performance of the endoscope handling service from when an endoscopy is started to when washing of the endoscope 24 by the washer 52 is ended. Therefore, according to the present configuration, it is possible for the user or the like to estimate the tight status of the performance of the endoscope handling service from when an endoscopy is started to when washing of the endoscope 24 by the washer 52 is ended, by referring to the operation rate of the endoscope 24.

Further, the standard operation time, during which the endoscope 24 used in the endoscope handling service is being operated, is an average value of the operation time of all the endoscopes 24 on the peak operation date. Therefore, according to the present configuration, an appropriate endoscope operation rate reference value can be calculated for comparison with each operation rate of all the endoscopes 24, as compared with the case of calculating the standard operation time by using only the operation time of one endoscope 24 on the peak operation date.

In the present second embodiment described above, although an example of the embodiment has been described in which the endoscope quantity specification information (hereinafter, also referred to as “first quantity specification information”), which can specify the quantity of the endoscopes 24 of which the operation rate is within the operation rate range equal to or greater than the endoscope operation rate reference value, is included in the medical service support information, the present disclosed technology is not limited to this. For example, the medical service support information may include notification information for notifying that the operation status of the endoscope 24 is tight, together with the first quantity specification information, or instead of the first quantity specification information.

For example, the notification information is information that includes a tight time slot, which is a time slot where the operation status of the endoscope 24 is tight (here, for example, the examination time slot), and second quantity specification information. The second quantity specification information refers to information that is capable of specifying the quantity of the endoscopes 24 operating during the tight time slot. In a case where the medical service support information includes the notification information, the tight time slot and the second quantity specification information are presented by the presentation device in a comparable state.

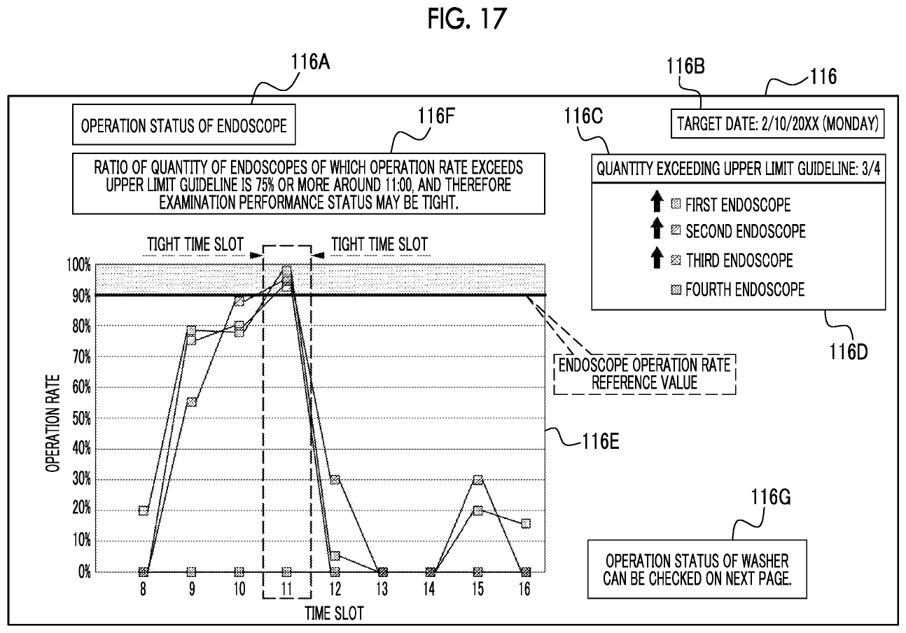

For example, as shown in FIG. 17, on the examination support graph 116E of the medical service support screen 116, the tight time slot and the quantity specified based on the second quantity specification information, that is, the quantity of the endoscopes 24 operating during the tight time slot, are displayed in a comparable state. The tight time slot is displayed in a manner distinguishable from other regions in the examination support graph 116E. Further, on the examination support graph 116E, three marks, which indicate the first to third endoscopes having operation rates exceeding the endoscope operation rate reference value, are plotted in the tight time slot.

Further, in the excess quantity display field 116C of the medical service support screen 116 shown in FIG. 17, a comment is displayed indicating that the quantity of the endoscopes 24 of which the operation rate exceeds the endoscope operation rate reference value as the upper limit guideline is three out of four. Further, in the examination support comment display field 116F of the medical service support screen 116 shown in FIG. 17, a comment is displayed indicating the tight status of the operation status of the endoscope 24. Specifically, a comment indicating the tight time slot and the tight status degree of the endoscopy is displayed in the examination support comment display field 116F. In the example shown in FIG. 17, in the examination support comment display field 116F, a comment such as “The ratio of the quantity of the endoscopes of which the operation rate exceeds the upper limit guideline is 75% or more around 11:00, and therefore, the examination performance status may be tight” is displayed.

In this way, by including the notification information for notifying that the operation status of the endoscope 24 is tight in the medical service support information, it is possible for the user or the like to recognize that the operation status of the endoscope 24 is tight. Further, the notification information is information that includes the tight time slot and the second quantity specification information, and the tight time slot and the second quantity specification information are presented by the presentation device in a comparable state, and therefore, it is possible for the user or the like to recognize the relationship between the tight time slot and the quantity of the endoscopes 24 operating during the tight time slot.

In the second embodiment described above, although an example of the embodiment has been described in which the endoscope operation rate reference value is derived regardless of the type of endoscopy, and the medical service support information is output regardless of the type of endoscopy, the present disclosed technology is not limited to this. For example, the CPU 98 of the support device server 74 may derive the endoscope operation rate reference value for each type of endoscopy and output the medical service support information for each type of endoscopy.

In this case, each of the processes of step ST206 to step ST308 of the medical service support process shown in FIG. 15 is performed for each endoscopy name based on the support device management database 113 in which the endoscopy names are associated with the examination start time point, the examination end time point, the washing start time point, and the washing end time point. Further, on the display 72, the medical service support screen 116 (see FIGS. 16 and 17) is displayed in a manner distinguishable for each endoscopy name. For example, the endoscopy name is displayed in the medical service support screen 116. Accordingly, it is possible to specify which type of endoscopy the medical service support screen 116 displayed on the display 72 is.

As described above, in the present example, the medical service support information is transmitted from the medical service support device 20 to the endoscope part management device 12 for each type of endoscopy, and the medical service support screen 116, which is based on the medical service support information, is displayed on the display 72 for each type of endoscopy. Therefore, according to the present example, it is possible to support the efficient performance of the endoscope handling service for each type of endoscopy.

In the second embodiment described above, although a broken line graph is exemplified as the examination support graph 116E, the present disclosed technology is not limited to this, and other graphs such as a bar graph, a pie chart, and a band graph may be used, and figures other than graphs such as a bubble chart may be used.

In the second embodiment described above, although the operation rate range that is equal to or greater than the endoscope operation rate reference value is exemplified, the present disclosed technology is not limited to this, and, for example, an operation rate range that is equal to or less than the endoscope operation rate reference value may be applied, and any operation rate range defined based on the endoscope operation rate reference value may be used. In this case as well, similarly to the second embodiment, the relationship between the operation rate range, which is defined based on the endoscope operation rate reference value, and the operation rate of the endoscope 24 may be visualized by using at least one of a graph, a comment, or a table and presented to the user or the like via the display 72.

In the second embodiment described above, although the endoscope operation rate reference value is exemplified as an upper limit guideline, the present disclosed technology is not limited to this. For example, the endoscope operation rate reference value may be an average operation rate of a plurality of endoscopes 24 (for example, all the endoscopes 24) in a period designated by the user or the like or may be a median value of the operation rates. Accordingly, it is possible for the user or the like to ascertain the correlation between the median value of the average operation rate or the operation rate, and the operation rate of each endoscope 24. The endoscope operation rate reference value is not limited to the average value and the median value and may be other statistical values such as the maximum value, the minimum value, or the most frequent value.

In the second embodiment described above, although an example of the embodiment has been described in which a comment such as “The number of examinations may be increased because the number of endoscope of which the operation rate exceeds the upper limit guideline is half or less” is displayed in the examination support comment display field 116F, the present disclosed technology is not limited to this. For example, in a case where the operation rate of all the endoscopes 24 exceeds the upper limit guideline, a message (a message expressing that the operation status of the endoscope 24 is tight) such as “The endoscope has no operating capacity” may be displayed in the examination support comment display field 116F, and in a case where the operation rate of at least one endoscope 24 is equal to or less than the upper limit guideline, a message (a message expressing that the operation of the endoscope 24 is not tight) such as “The endoscope has operating capacity” may be displayed in the examination support comment display field 116F.

Further, in the medical service support screen 116, a symbol mark for each of the first to fourth endoscopes and marks such as “A”, “B”, and “C” that are selectively assigned to each symbol mark may be displayed. The “A” mark is a mark indicating that the endoscope 24 has operating capacity, the “B” mark is a mark indicating that the endoscope 24 has a small amount of operating capacity, and the “C” mark is a mark indicating that the endoscope 24 has no operating capacity. Further, the tightness degree may be visualized by changing a display aspect such as a color and/or a font of each of the symbol marks of the first to fourth endoscopes to highlight them.

In the medical service support process (see FIG. 15) according to the second embodiment, although the peak operation date is derived by executing the process of step ST206, the present disclosed technology is not limited to this. For example, the derivation unit 98E may derive a date on which the operation status of the endoscope 24 peaks as the peak operation date with reference to the support device management database 113 (see FIG. 9). The peak operation date in this case indicates a date on which the operation status of the endoscope 24 peaks within a period designated in advance (for example, a period designated by the user or the like). Examples of the peak operation date in this case include a date on which the endoscope 24 is used the highest number of times. The date on which the endoscope 24 is used can be specified based on the examination start time point, the examination end time point, the washing start time point, or the washing end time point stored in the support device management database 113. Further, other examples of the peak operation date include a date on which the number of times the endoscope standby time falls below a threshold value (for example, 50 minutes) is equal to or greater than the reference number of times.

### Third Embodiment

In the first embodiment described above, although an example of the embodiment has been described in which the performance of the endoscope handling service is supported by using the medical service support information obtained based on the operation rate of the information processing apparatus 36, which is an example of the “medical device” according to the present disclosed technology, in the third embodiment, a case will be described where the performance of the endoscope handling service is supported by using the medical service support information obtained based on the operation rate of the washer 52. Further, in the present third embodiment, the same components as those in the first embodiment are designated by the same reference numerals, the description thereof will be omitted, and the parts different from those in the first embodiment will be described.

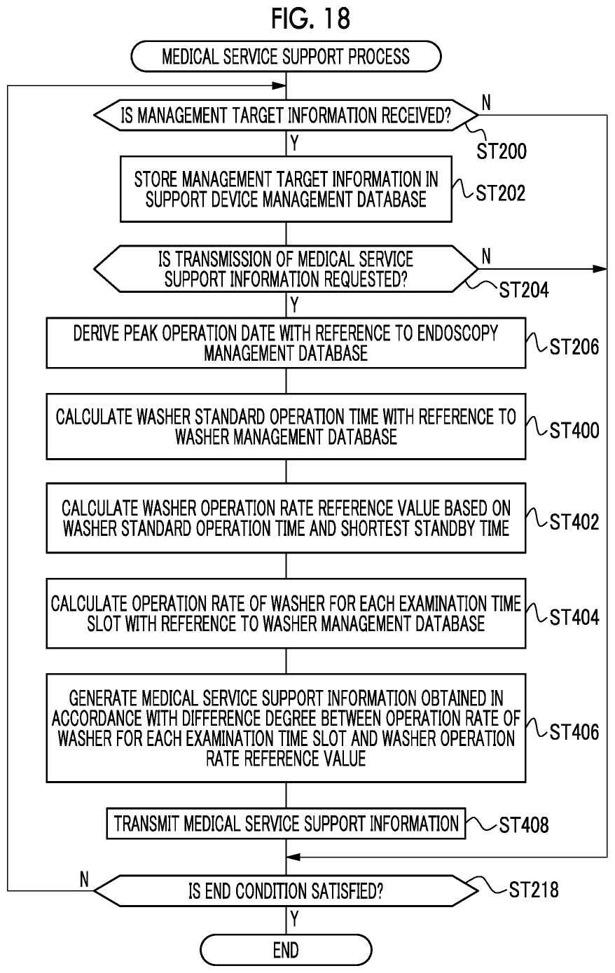

FIG. 18 shows an example of a flow of a medical service support process according to the present third embodiment.

The flowchart shown in FIG. 18 is different from the flowchart shown in FIG. 11 in that it includes step ST400 to step ST408 instead of step ST208 to step ST216. Therefore, in the following description, steps in the flowchart shown in FIG. 11 that are the same as those in the flowchart shown in FIG. 18 are given the same step numbers, the description thereof will be omitted, and steps different from those included in the flowchart shown in FIG. 11 will be described.

In the medical service support device shown in FIG. 18, in step ST400, the derivation unit 98E calculates washer standard operation time for the peak operation date derived in step ST206 with reference to the washer management database 112. After the process in step ST400 is executed, the medical service support process proceeds to step ST402.

In step ST400, the washer standard operation time is a statistical value of time based on the operation time of the washer 52 on the peak operation date, that is, the operation time of all washers 52 (washer operation time for all the washers 52) on the peak operation date. Here, the washer operation time refers to the operation time of the washer 52. The washer operation time is an example of the “operation time of the washer” according to the present disclosed technology and is time from when the washing start button 60A is turned on in step ST28 shown in FIG. 2 to when the washing of the endoscope 24 by the washer 52 is ended in step ST32 shown in FIG. 2.

For example, the washer standard operation time is an average value of operation time of all the washers 52 on the peak operation date. The operation time of the washer 52 is specified based on the washing start time point and the washing end time point stored in the washer management database 112.

Here, although the average value of the operation time of all the washers 52 is exemplified as the washer standard operation time, this is merely an example, and it may be a statistical value such as a median value, the most frequent value, the maximum value, the minimum value, or a percentile of the operation time of all the washers 52. Further, the washer standard operation time may be calculated within a time range (for example, within a range of substantially 10 minutes to 50 minutes) predetermined with an upper limit value and a lower limit value.

In step ST402, the derivation unit 98E calculates a washer operation rate reference value based on the washer standard operation time calculated in step ST400 and on the shortest washer standby time. After the process in step ST402 is executed, the medical service support process proceeds to step ST404. In the present step ST402, the shortest washer standby time refers to the shortest standby time from when washing of the endoscope 24 by the washer 52 is ended to when washing of the endoscope 24 by the washer 52 is started. Here, the shortest standby time refers to, for example, the shortest time of the standby time from the end of washing performed by each of all the washers 52 to the start of washing. Here, as an example of the shortest standby time, predetermined time (for example, pre-designated time between 1 minute and 3 minutes) is applied as automatic washing time shown in FIG. 2, that is, the shortest time from when washing of the endoscope 24 by the washer 52 is ended (for example, from when the process of step ST32 shown in FIG. 2 is ended) to when washing of the endoscope 24 by the washer 52 is started (for example, to when the process of step ST28 shown in FIG. 2 is started again). The shortest washer standby time may be a fixed value or a variable value that is changed according to an instruction received by the reception device 70 or the like.

In step ST402, the washer operation rate reference value is a value based on a ratio of the washer standard operation time with respect to time including the washer standard operation time calculated in step ST400 and the shortest washer standby time. Examples of the washer operation rate reference value include a value obtained by multiplying the ratio of the washer standard operation time with respect to the sum of the washer standard operation time calculated in step ST400 and the shortest washer standby time, by an adjustment factor. Examples of the adjustment factor according to the present third embodiment include 0.9. The adjustment factor of “0.9” is merely an example, and the adjustment factor may be another decimal number less than 1. Further, the adjustment factor may be a fixed value or a variable value.

Further, although an example of the embodiment has been described in which the ratio of the washer standard operation time with respect to the sum of the washer standard operation time and the shortest washer standby time is multiplied by the adjustment factor, this is merely an example, and a value which is obtained by adding or subtracting an adjustment value to the ratio of the washer standard operation time with respect to the sum of the washer standard operation time and the shortest washer standby time may be used as the washer operation rate reference value.

In step ST404, the derivation unit 98E derives an operation rate of each washer 52 for each examination time slot on the peak operation date derived in step ST206 with reference to the washer management database 112. After the process in step ST404 is executed, the medical service support process proceeds to step ST406.

The operation rate of the washer 52 is calculated by the derivation unit 98E according to the expression “(operation time of washer 52)/{(operation time of washer 52)+(washer standby time)}” for each examination time slot on the peak operation date. The washer standby time included in this expression is an example of the “standby time” according to the present disclosed technology.

In step ST406, the generation unit 98F generates the medical service support information obtained in accordance with a difference degree between the operation rate of each washer 52 for each examination time slot on the peak operation date (the operation rate calculated in step ST404) and the washer operation rate reference value calculated in step ST402. After the process in step ST406 is executed, the medical service support process proceeds to step ST408.

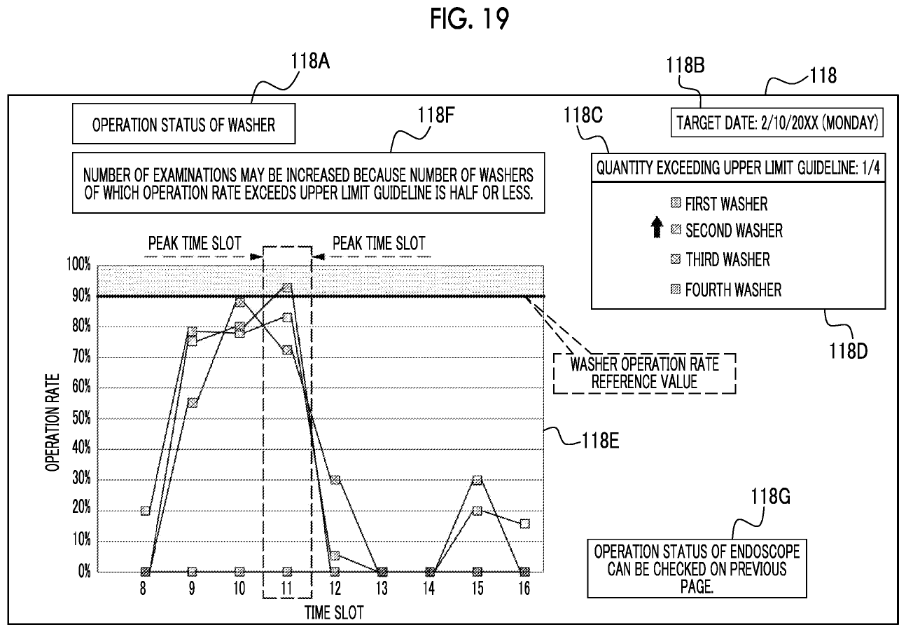

The medical service support information is information that is capable of being represented via a comment, a figure, or the like by a presentation device (here, the display 72 as an example) and includes screen information indicating a medical service support screen 118 (see FIG. 19). As described in detail later, the medical service support screen 118 includes a graph, a comment, and the like that visualize the difference degree between the operation rate of each washer 52 for each examination time slot on the peak operation date and the washer operation rate reference value. The difference degree between the operation rate of the washer 52 and the washer operation rate reference value indicates a degree to which the operation rate of the washer 52 deviates from the washer operation rate reference value, and corresponds to, for example, a value obtained by subtracting the operation rate of the washer 52 from the washer operation rate reference value, or to the ratio of the operation rate of the washer 52 to the washer operation rate reference value.

Further, the medical service support information includes, as the screen information, washer related information related to the washer 52 of which the operation rate is within an operation rate range defined based on the washer operation rate reference value. The washer related information is an example of the “medical device related information” according to the present disclosed technology. Further, in the present third embodiment, as an example of the operation rate range defined based on the washer operation rate reference value, the operation rate range equal to or greater than the washer operation rate reference value is applied. Further, in the present third embodiment, the endoscope ID is applied as an example of the endoscope related information. As described in detail later, on the medical service support screen 118, the washer 52 of which the operation rate is within the operation rate range equal to or greater than the washer operation rate reference value is visualized and displayed so as to be specifiable.

Further, in the washer related information, washer quantity specification information (an example of the medical device quantity specification information) that is capable of specifying a quantity of the washers of which the operation rate is within the operation rate range equal to or greater than the washer operation rate reference value is included. As described in detail later, on the medical service support screen 118, the quantity of the washers 52 of which the operation rate is within the operation rate range equal to or greater than the washer operation rate reference value is visualized and displayed so as to be specifiable.

In step ST408, the transmission unit 98B transmits the medical service support information, which is generated in step ST406, to the endoscope part management device main body 68 via the communication I/F 104 (see FIG. 7). After the process in step ST408 is executed, the medical service support process proceeds to step ST218.

In a case where the medical service support information is transmitted by executing the process of step ST408, as described above, the medical service support information is received through the support device communication I/F 84 of the endoscope part management device 12 (see step ST110 shown in FIG. 10). As shown in step ST112 shown in FIG. 10, the display control unit 78D of the endoscope part management device 12 generates the medical service support screen 118 based on the medical service support information, and the generated medical service support screen 118 is displayed on the display 72 (FIGS. 1 and 3) (see FIG. 19).

In the following, in the present third embodiment, for convenience of explanation, a case will be described where operation statuses of a first washer, a second washer, a third washer, and a fourth washer are presented, as the four washers 52, to the user or the like via the display 72 as the medical service support screen 118.

As an example, as shown in FIG. 19, on the medical service support screen 118, a screen title display field 118A, an operation rate calculation target date display field 118B, an excess quantity display field 118C, an explanatory note display field 118D, an examination support graph 118E, an examination support comment display field 118F, and a guidance comment display field 118G are displayed.

In the screen title display field 118A, a title that can be recognized as a screen showing the operation status of the washer 52 is displayed. In the example shown in FIG. 19, an “operation status of the washer” is displayed in the screen title display field 118A.

In the operation rate calculation target date display field 118B, a date, which is a calculation target of the operation rate in step ST404 included in the medical service support process shown in FIG. 18, that is, the peak operation date derived in step ST206 included in the medical service support process shown in FIG. 18, is displayed. In the example shown in FIG. 19, the peak operation date is represented by the date and day.

In the excess quantity display field 118C, the quantity of the washers 52 of which the operation rate exceeds the upper limit guideline is displayed. In the example shown in FIG. 19, in the excess quantity display field 118C, a comment such as “Quantity exceeding upper limit guideline: ¼” is displayed as the recognizable information that one washer 52 out of the four washers 52 exceeds the upper limit guideline. Here, the upper limit guideline refers to the washer operation rate reference value calculated by executing the process of step ST402 included in the medical service support process shown in FIG. 18. The upper limit guideline is not limited to this, and may be, for example, a value obtained by finely adjusting the washer operation rate reference value.

In the explanatory note display field 118D, an explanatory note, which is related to the examination support graph 118E, is displayed. In the example shown in FIG. 19, in the explanatory note display field 118D, an explanatory note of the first washer, an explanatory note of the second washer, an explanatory note of the third washer, and an explanatory note of the fourth washer are displayed. Further, in the example shown in FIG. 19, in the explanatory note display field 118D, an upward arrow, which indicates that the operation rate of the second washer exceeds the upper limit guideline, is also displayed.

The examination support graph 118E is an example of a “figure” according to the present disclosed technology. The examination support graph 118E is a graph showing a time change of the operation rate calculated by executing the process of step ST404 included in the medical service support process shown in FIG. 18. A horizontal axis of the examination support graph 118E is the examination time slot of the peak operation date, and a vertical axis of the examination support graph 118E is the operation rate of the washer 52.

In the examination support graph 118E, it is shown that the operation rate of the second washer around 11:00 exceeds the washer operation rate reference value as the upper limit guideline, and in other time slots, none of the operation rates of the first to fourth washers reach the washer operation rate reference value. Further, in the examination support graph 118E, it is shown that the difference degree between each of the operation rates of the first to fourth washers and the washer operation rate reference value is visualized for each examination time slot.

Further, in the examination support graph 118E, the operation rate range exceeding the upper limit guideline of the operation rate, that is, the operation rate range equal to or greater than the washer operation rate reference value, is displayed in a manner distinguishable from other regions. Further, in the examination support graph 118E, the range indicating a peak time slot is displayed in a manner distinguishable from other regions. The peak time slot refers to an examination time slot in which the average value of the operation rates of the washer 52 is the highest among the examination time slots. In the example shown in FIG. 19, around 11:00 is displayed as the peak time slot in a manner distinguishable from other regions (in the example shown in FIG. 19, a broken line frame).

In the examination support comment display field 118F, a comment contributing to performing an endoscopy is displayed. Specifically, a comment which indicates a correlation between the quantity of the washers 52 of which the operation rate is within the operation rate range equal to or greater than the washer operation rate reference value as the upper limit guideline and the number of examinations (for example, the number of times an endoscopy can be performed) is displayed in the examination support comment display field 118F. In the example shown in FIG. 19, in the examination support comment display field 118F, a comment such as “The number of examinations may be increased because the number of washers of which the operation rate exceeds the upper limit guideline is half or less” is displayed. The comment, which is displayed in the examination support comment display field 118F, is an example of a “comment” according to the present disclosed technology.

In the guidance comment display field 118G, a guidance comment is displayed. In the example shown in FIG. 19, in the guidance comment display field 118G, a comment is displayed indicating that the operation status of the endoscope 24 can be checked on the previous page of the medical service support screen 118. Here, although a comment which indicates that the operation status of the endoscope 24 can be checked on the previous page of the medical service support screen 118 is shown as an example, the present disclosure is not limited to this, and it may be a comment indicating a page of the medical service support screen 118 and a medical device where the operation status can be checked.

As described above, in the medical service support device 20 according to the present third embodiment, the washer operation rate reference value is derived based on the standard operation time during which the washer 52 used in the endoscope handling service is being operated and on the washer standby time. In the medical service support device 20, the medical service support information, which is obtained in accordance with the difference degree between the operation rate of the washer 52 and the washer operation rate reference value, is transmitted to the endoscope part management device 12. In the endoscope part management device 12, the medical service support screen 118 is generated based on the medical service support information and is displayed on the display 72. Since the medical service support screen 118 is presented to the user or the like in a state in which the medical service support information is visualized, the user or the like ascertains the difference degree between the operation rate of the washer 52 and the washer operation rate reference value. Therefore, according to the present configuration, it is possible to support the efficient performance of the endoscope handling service.

Further, the medical service support information includes information related to the washer 52 of which the operation rate is within the operation rate range equal to or greater than the washer operation rate reference value. In the endoscope part management device 12, the medical service support screen 118 is generated based on the medical service support information and is displayed on the display 72. On the medical service support screen 118, since the information (for example, the examination support graph 118E) which is related to the washer 52 of which the operation rate is within the operation rate range equal to or greater than the washer operation rate reference value is presented to the user in a visualized state, the user or the like ascertains the information that is related to the washer 52 of which the operation rate is within the operation rate range equal to or greater than the washer operation rate reference value. Therefore, according to the present configuration, the user or the like can accurately ascertain the information related to the washer 52 of which the operation rate is within the operation rate range equal to or greater than the washer operation rate reference value, as compared with the case where the medical service support information includes only the operation rate of the washer 52.

Further, the medical service support information includes information related to the washer 52 of which the operation rate is within the operation rate range equal to or greater than the washer operation rate reference value on the peak operation date. In the endoscope part management device 12, the medical service support screen 118 is generated based on the medical service support information and is displayed on the display 72. On the medical service support screen 118, since the information (for example, the examination support graph 118E) which is related to the washer 52 of which the operation rate is within the operation rate range equal to or greater than the washer operation rate reference value on the peak operation date is presented to the user in a visualized state, the user or the like ascertains the information that is related to the washer 52 of which the operation rate is within the operation rate range equal to or greater than the washer operation rate reference value on the peak operation date. Therefore, according to the present configuration, the user or the like can accurately ascertain the information related to the washer 52 of which the operation rate is within the operation rate range equal to or greater than the washer operation rate reference value on the peak operation date, as compared with the case where the medical service support information includes only the operation rate of the washer 52.

Further, the peak operation date is a date on which an endoscopy is performed the highest number of times. Therefore, according to the present configuration, the user or the like can accurately ascertain the information related to the washer 52 of which the operation rate is within the operation rate range equal to or greater than the washer operation rate reference value on the date on which an endoscopy is performed the highest number of times, as compared with the case where the medical service support information includes only the operation rate of the washer 52.

Further, in the medical service support information, washer quantity specification information that is capable of specifying a quantity of the washers 52 of which the operation rate is within the operation rate range equal to or greater than the washer operation rate reference value is included as the information related to the washer 52. On the medical service support screen 118, the quantity of the washers 52 of which the operation rate is within the operation rate range equal to or greater than the washer operation rate reference value is visualized and displayed so as to be specifiable. Therefore, according to the present configuration, the user or the like can ascertain the quantity of the washers 52 of which the operation rate is within the operation rate range equal to or greater than the washer operation rate reference value.

Further, the standard operation time, during which the washer 52 used in the endoscope handling service is being operated, is time based on the operation time of all the washers 52 on the peak operation date. In the medical service support device 20, the washer operation rate reference value is derived based on the standard operation time during which the washer 52 is being operated and on the washer standby time. Therefore, according to the present configuration, a value with high reliability can be obtained as the washer operation rate reference value on the peak operation date, as compared with a case where the standard operation time, during which the washer 52 used in the endoscope handling service is being operated, is defined as time based on the operation time of all the washers 52 during time periods other than the peak operation date.

Further, the washer operation rate reference value is a value based on a ratio of the washer standard operation time with respect to the sum of the shortest washer standby time defined as the shortest time of the washer standby time and the washer standard operation time. Therefore, according to the present configuration, it is possible to increase the washer rate reference value, which is the target for comparison of the operation rate of the washer 52, as compared with the case where the ratio of the washer standard operation time with respect to the sum of the time longer than the shortest washer standby time and the washer standard operation time is used as the washer operation rate reference value.

Further, the washer operation rate reference value is a value obtained by multiplying the ratio of the washer standard operation time with respect to the sum of the shortest washer standby time, which is defined as the shortest washer standby time, and the washer standard operation time, by the adjustment factor. For example, the adjustment factor is defined according to an external factor (for example, rest time for endoscopy workers and/or potential problems) that affects at least one of the shortest washer standby time or the washer standard operation time. Therefore, according to the present configuration, an appropriate value can be obtained as the washer operation rate reference value, which is the target for comparison of the operation rate of the washer 52, even in a case where there is an external factor that affects at least one of the shortest washer standby time or the washer standard operation time, as compared with the case where only the ratio of the washer standard operation time with respect to the sum of the shortest washer standby time and the washer standard operation time is used as the washer operation rate reference value.

Further, the operation rate of the washer 52 is calculated based on the washer standby time (see FIG. 2) and on the operation time of the washer 52. The operation time of the washer 52 is time from when the washer 52 starts washing the endoscope 24 to when the washer 52 finishes washing the endoscope 24. That is, the operation time of the washer 52 represents a tight status (for example, the tight status of the operation of the washer 52) degree of the performance of the endoscope handling service from when the washer 52 starts washing the endoscope 24 to when the washer 52 finishes washing the endoscope 24. Therefore, according to the present configuration, it is possible for the user or the like to estimate the tight status (for example, the tight status of the operation of the washer 52) of the performance of the endoscope handling service from when the washer 52 starts washing the endoscope 24 to when the washer 52 finishes washing the endoscope 24 by referring to the operation rate of the washer 52.

Further, the standard operation time, during which the washer 52 used in the endoscope handling service is being operated, is an average value of the operation time of all the washers 52 on the peak operation date. Therefore, according to the present configuration, an appropriate washer operation rate reference value can be calculated for comparison with each operation rate of all the washers 52, as compared with the case of calculating the standard operation time by using only the operation time of one washer 52 on the peak operation date.

In the present third embodiment described above, although an example of the embodiment has been described in which the washer quantity specification information (hereinafter, also referred to as “third quantity specification information”), which can specify the quantity of the washers 52 of which the operation rate is within the operation rate range equal to or greater than the washer operation rate reference value, is included in the medical service support information, the present disclosed technology is not limited to this. For example, the medical service support information may include notification information for notifying that the operation status of the washer 52 is tight, together with the third quantity specification information, or instead of the third quantity specification information.

For example, the notification information is information that includes a tight time slot, which is a time slot where the operation status of the washer 52 is tight (here, for example, the examination time slot), and fourth quantity specification information. The fourth quantity specification information refers to information that is capable of specifying the quantity of the washers 52 operating during the tight time slot. In a case where the medical service support information includes the notification information, the tight time slot and the fourth quantity specification information are presented by the presentation device in a comparable state.

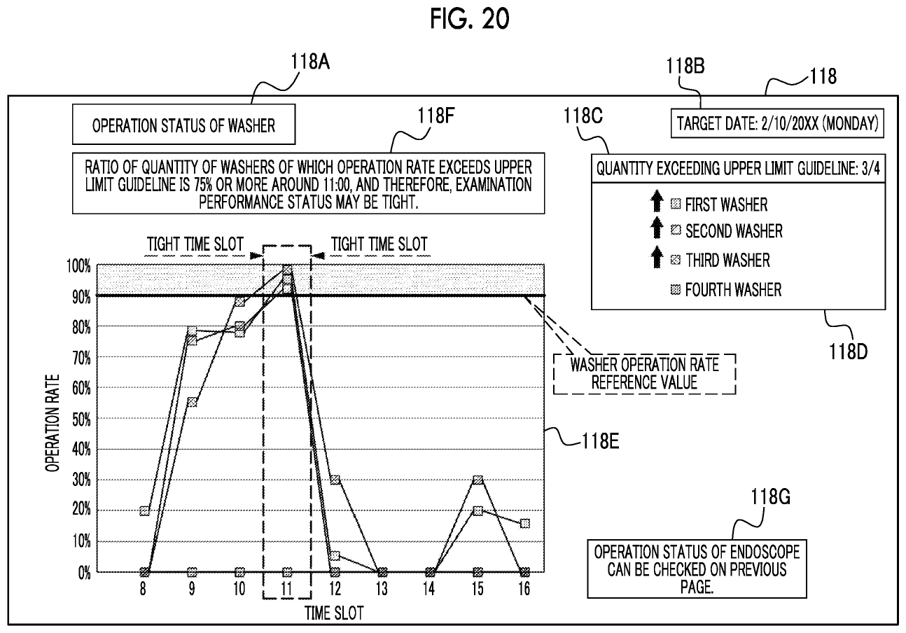

For example, as shown in FIG. 20, on the examination support graph 118E of the medical service support screen 118, the tight time slot and the quantity specified based on the fourth quantity specification information, that is, the quantity of the washers 52 operating during the tight time slot, are displayed in a comparable state. The tight time slot is displayed in a manner distinguishable from other regions in the examination support graph 118E. Further, on the examination support graph 118E, three marks, which indicate the first to third washers having operation rates exceeding the washer operation rate reference value, are plotted in the tight time slot.

Further, in the excess quantity display field 118C of the medical service support screen 118 shown in FIG. 20, a comment is displayed indicating that the quantity of the washers 52 of which the operation rate exceeds the washer operation rate reference value as the upper limit guideline is three out of four. Further, in the examination support comment display field 118F of the medical service support screen 118 shown in FIG. 20, a comment is displayed indicating the tight status of the operation status of the washer 52. Specifically, a comment indicating the tight time slot and the tight status degree of the endoscopy is displayed in the examination support comment display field 118F. In the example shown in FIG. 20, in the examination support comment display field 118F, a comment such as “The ratio of the quantity of the washers of which the operation rate exceeds the upper limit guideline is 75% or more around 11:00, and therefore, the examination performance status may be tight” is displayed.

In this way, by including the notification information for notifying that the operation status of the washer 52 is tight in the medical service support information, it is possible for the user or the like to recognize that the operation status of the washer 52 is tight. Further, the notification information is information that includes the tight time slot and the fourth quantity specification information, and the tight time slot and the fourth quantity specification information are presented by the presentation device in a comparable state, and therefore, it is possible for the user or the like to recognize the relationship between the tight time slot and the quantity of the washers 52 operating during the tight time slot.

Further, since the plurality of washers 52 include a single-endoscope washer and a dual-endoscope washer, it is also conceivable that the performance status of the endoscopy is tight due to the fact that the dual-endoscope washer is not used efficiently. For example, it is conceivable that the performance status of the endoscopy is tight due to the fact that the dual-endoscope washer washes only one endoscope 24 and the other endoscopes 24 are in a washing wait state, or conversely, washing by the dual-endoscope washer is not started until two endoscopes 24 are inserted into the dual-endoscope washer, even though it is at a time when two endoscopes 24 can be inserted at the same time.

Such a status is specified by the CPU 98 of the support device server 74 from the washing information 66 stored in the washer management database 112. In a case where there is a possibility that the performance status of endoscopy is tight due to the fact that the dual-endoscope washer is not used efficiently, the notification information may include information for notifying that there is a possibility that the performance status of the endoscopy is tight due to the dual-endoscope washer. In this case, for example, a message indicating that there is a possibility that the performance status of the endoscopy is tight due to the fact that the dual-endoscope washer is not used efficiently may be displayed in the examination support comment display field 118F, instead of the above-described comment or together with the above-described comment.

In the third embodiment described above, although an example of the embodiment has been described in which the washer operation rate reference value is derived regardless of the type of endoscopy, and the medical service support information is output regardless of the type of endoscopy, the present disclosed technology is not limited to this. For example, the CPU 98 of the support device server 74 may derive the washer operation rate reference value for each type of endoscopy and output the medical service support information for each type of endoscopy.

In this case, each of the processes of step ST206 to step ST408 of the medical service support process shown in FIG. 18 is performed for each endoscopy name based on the support device management database 113 in which the endoscopy names are associated with the examination start time point, the examination end time point, the washing start time point, and the washing end time point. Further, on the display 72, the medical service support screen 118 (see FIGS. 19 and 20) is displayed in a manner distinguishable for each endoscopy name. For example, the endoscopy name is displayed in the medical service support screen 118. Accordingly, it is possible to specify which type of endoscopy the medical service support screen 118 displayed on the display 72 is.

As described above, in the present example, the medical service support information is transmitted from the medical service support device 20 to the endoscope part management device 12 for each type of endoscopy, and the medical service support screen 118, which is based on the medical service support information, is displayed on the display 72 for each type of endoscopy. Therefore, according to the present example, it is possible to support the efficient performance of the endoscope handling service for each type of endoscopy.

In the third embodiment described above, although a broken line graph is exemplified as the examination support graph 118E, the present disclosed technology is not limited to this, and other graphs such as a bar graph, a pie chart, and a band graph may be used, and figures other than graphs such as a bubble chart may be used.

In the third embodiment described above, although the operation rate range that is equal to or greater than the washer operation rate reference value is exemplified, the present disclosed technology is not limited to this, and, for example, an operation rate range that is equal to or less than the washer operation rate reference value may be applied, and any operation rate range defined based on the washer operation rate reference value may be used. In this case as well, similarly to the first embodiment, the relationship between the operation rate range, which is defined based on the washer operation rate reference value, and the operation rate of the washer 52 may be visualized by using at least one of a graph, a comment, or a table and presented to the user or the like via the display 72.

In the medical service support process (see FIG. 18) according to the third embodiment, although the peak operation date is derived by executing the process of step ST206, the present disclosed technology is not limited to this. For example, the derivation unit 98E may derive a date on which the operation status of the washer 52 peaks as the peak operation date with reference to the washer management database 112 (see FIG. 9). The peak operation date in this case indicates a date on which the operation status of all the washers 52 installed in the washer room 50 peaks within a period designated in advance (for example, a period designated by the user or the like). Examples of the peak operation date in this case include a date on which the washer 52 is used the highest number of times. The date on which the washer 52 is used can be specified based on the washing start time point or the washing end time point of the washing information 66 stored in the washer management database 112. Further, other examples of the peak operation date include a date on which the number of times the washer standby time (see FIG. 2) falls below a threshold value (for example, 50 minutes) is equal to or greater than the reference number of times.

In the third embodiment described above, although the washer operation rate reference value is exemplified as an upper limit guideline, the present disclosed technology is not limited to this. For example, the washer operation rate reference value may be an average operation rate of a plurality of washers 52 (for example, all the washers 52) in a period designated by the user or the like or may be a median value of the operation rates. Accordingly, it is possible for the user or the like to ascertain the correlation between the median value of the average operation rate or the operation rate, and the operation rate of each washer 52. The washer operation rate reference value is not limited to the average value and the median value and may be other statistical values such as the maximum value, the minimum value, or the most frequent value.

In the third embodiment described above, although an example of the embodiment has been described in which a comment such as “The number of examinations may be increased because the number of washers of which the operation rate exceeds the upper limit guideline is half or less” is displayed in the examination support comment display field 118F, the present disclosed technology is not limited to this. For example, in a case where the operation rate of all the washers 52 exceeds the upper limit guideline, a message (a message expressing that the operation status of the washer 52 is tight) such as “The washer has no operating capacity” may be displayed in the examination support comment display field 114F, and in a case where the operation rate of at least one washer 52 is equal to or less than the upper limit guideline, a message (a message expressing that the operation status of the washer 52 is not tight) such as “The washer has operating capacity” may be displayed in the examination support comment display field 118F.

Further, in the medical service support screen 118, a symbol mark for each of the first to fourth washers and marks such as “A”, “B”, and “C” that are selectively assigned to each symbol mark may be displayed. The “A” mark is a mark indicating that the washer 52 has operating capacity, the “B” mark is a mark indicating that the washer 52 has a small amount of operating capacity, and the “C” mark is a mark indicating that the washer 52 has no operating capacity. Further, the tightness degree may be visualized by changing a display aspect such as a color and/or a font of each of the symbol marks of the first to fourth washers to highlight them.

In each of the embodiments described above, although an example of the embodiment has been described in which whether or not the operation rate of the medical device exceeds the operation rate reference value is presented to the user or the like by the presentation device (for example, the display 72), the present disclosed technology is not limited to this. For example, information which is related to the medical device of which the operation rate falls below an operation rate reference value (for example, the quantity of medical devices and/or a medical device ID), information which is related to the medical device of which the operation rate coincides with an operation rate reference value, and/or information with which it is possible to ascertain how much the operation rate deviates from an operation rate reference value (for example, a difference between an operation rate and an operation rate reference value and/or a ratio of an operation rate to an operation rate reference value) may be visualized and/or audibly presented by the presentation device.

In each of the embodiments described above, although an example of the embodiment has been described in which information (for example, an explanatory note) that is capable of specifying a medical device is displayed on the medical service support screens 114, 116, and 118, the present disclosed technology is not limited to this, and other information related to a medical device, such as a manufacturing time period of the medical device and/or the number of cumulative uses of the medical device, may be displayed on the medical service support screens 114, 116, and 118, instead of the information that is capable of specifying the medical device or together with the information that is capable of specifying the medical device.

In each of the embodiments described above, although an example of the embodiment has been described in which the medical service support information is generated based on the operation time, the standby time, and the operation rate in a case where an endoscopy is performed in units of the examination rooms 30, the present disclosed technology is not limited to this, and the medical service support information may be generated based on operation time, standby time, and an operation rate in a case where an endoscopy is performed in units of clinical departments (for example, internal medicine or surgery) or medical institutions (for example, clinic or general hospital).

In each of the embodiments described above, although the examination room 30 has been exemplified as an example, it does not necessarily have to be one room, and may be a space obtained by dividing one room by a curtain and/or a stand or the like.

In the above embodiment, although the endoscope processor device 32 and the information processing apparatus 36 are separate units, the present disclosed technology is not limited to this, and the endoscope processor device 32 and the information processing apparatus 36 may be integrated. In this case, for example, a device corresponding to the information processing apparatus 36 may be incorporated into the endoscope processor device 32, or a device corresponding to the endoscope processor device 32 may be incorporated into the information processing apparatus main body 38.

In each of the embodiments described above, although an example of the embodiment has been described in which the medical service support information is visualized and presented to the user or the like by displaying the medical service support screens 114, 116, and 118 on the display 72, the present disclosed technology is not limited to this. For example, the medical service support information may be audibly output by a sound reproducing device, or the medical service support information may be printed on a recording medium (for example, paper) by a printer and output, instead of or together with the visual presentation by the display 72 described above.

In each of the embodiments described above, although the endoscope handling service including an endoscopy, which is an example of the “endoscope procedure service” according to the present disclosed technology, has been described, the present disclosed technology is not limited to this, and a medical device handling service including a medical device procedure service other than an endoscopy may be used. For example, the endoscope procedure service may be a service including endoscopic surgery and/or endoscopic treatment. Examples of endoscopic surgery include laparoscopic surgery and/or treatment, thoracoscopic surgery and/or treatment, cystoscopic surgery and/or treatment, choledoscopic surgery and/or treatment, spinal endoscopy and/or therapy, angioscopy surgery and/or treatment, and epidural endoscopic surgery and/or treatment.

In each of the embodiments described above, although the endoscope 24, the information processing apparatus 36, and the washer 52 are described as examples of the medical device, the present disclosed technology is not limited to this, and the medical device may be a medical device other than the endoscope 24, the information processing apparatus 36, and the washer 52. Examples of the medical device other than the endoscope 24, the information processing apparatus 36, and the washer 52 include a medical management device (for example, a device having a processor and a memory) corresponding to the information processing apparatus 36, a medical accessory device that is attachably and detachably connected to a medical management device in the same way that the endoscope 24 is attachably and detachably connected to the information processing apparatus 36 (for example, replaceable ultrasonic probes and/or replaceable therapeutic tools used in dental treatment, or the like), and a washer that washes the medical accessory device similar to the washer 52 that washes the endoscope 24.

In each of the embodiments described above, although the barcode 42 and the subject barcode are exemplified, another two-dimensional code such as a quick response (QR) code (registered trademark) may be used, or a noncontact storage medium using radio frequency identification (RFID) technology may be used, instead of at least one of the barcode 42 or the subject barcode. Further, in this case, the endoscope 24 or a device that can acquire information which is capable of specifying a subject may be applied by recognizing these two-dimensional codes and/or noncontact storage media instead of the barcode readers 40 and 64.

In each of the embodiments described above, although the medical service support device 20 is exemplified, a computer, which is used together with the endoscope part management device 12 on-premises, may be caused to execute the medical service support processing program 108 (see FIG. 8) instead of the medical service support device 20. Further, the management information storage device 76 may also be used on-premises together with the endoscope part management device 12.

Further, in each of the embodiments described above, although an example of the embodiment has been described in which the medical service support processing program 108 is stored in the storage 100, the medical service support processing program 108 may be stored in any portable storage medium such as an SSD or universal serial bus (USB) memory, or the medical service support processing program 108 may be stored in a non-temporary storage medium. The medical service support processing program 108 stored in the non-temporary storage medium is installed, for example, in the support device server 74 or the like.

Further, in each of the embodiments described above, although the support device server 74 including the CPU 98, the storage 100, and the memory 102 has been exemplified, the present disclosed technology is not limited to this, and a device including an application specific integrated circuit (ASIC), a field-programmable gate array (FPGA), and/or a programmable logic device (PLD) may be applied instead of the support device server 74 or together with the support device server 74.

As a hardware resource for executing the medical service support process described in the above embodiment, the following various processors can be used. Examples of the processor include a CPU, which is a general-purpose processor that functions as a hardware resource for executing the medical service support process by executing software, that is, a program. Further, examples of the processor include a dedicated electric circuit, which is a processor having a circuit configuration specially designed for executing specific processing such as an FPGA, a PLD, or an ASIC. Any processor has a memory built in or connected to it, and any processor uses the memory to execute a terminal side process.

The hardware resource for executing the medical service support process may be configured with one of these various processors or may be configured with a combination (for example, a combination of a plurality of FPGAs or a combination of a CPU and an FPGA) of two or more processors of the same type or different types. Further, the hardware resource for executing the medical service support process may be one processor.

As an example of a configuration with one processor, first, one processor is configured with a combination of one or more CPUs and software, and there is an embodiment in which this processor functions as a hardware resource for executing the medical service support process. Secondly, as typified by a system-on-a-chip (SoC), there is an embodiment in which a processor that implements the functions of the entire system including a plurality of hardware resources for executing the medical service support process with one IC chip is used. As described above, the medical service support process is implemented by using one or more of the above-mentioned various processors as a hardware resource.

Further, as the hardware-like structure of these various processors, more specifically, an electric circuit in which circuit elements such as semiconductor elements are combined can be used. Further, the above-mentioned terminal side process is only an example. Therefore, it goes without saying that unnecessary steps may be deleted, new steps may be added, or the processing order may be changed within a range that does not deviate from the purpose.

The contents described above and the contents shown in the illustrations are detailed explanations of the parts related to the present disclosed technology and are merely an example of the present disclosed technology. For example, the description related to the configuration, function, action, and effect described above is an example related to the configuration, function, action, and effect of a portion according to the present disclosed technology. Therefore, it goes without saying that unnecessary parts may be deleted, new elements may be added, or replacements may be made to the contents described above and the contents shown in the illustrations, within the range that does not deviate from the purpose of the present disclosed technology. Further, in order to avoid complications and facilitate understanding of the parts of the present disclosed technology, in the contents described above and the contents shown in the illustrations, the descriptions related to common technical knowledge or the like that do not require special explanation in order to enable the implementation of the present disclosed technology are omitted.

In the present specification, “A and/or B” is synonymous with “at least one of A or B”. That is, “A and/or B” means that it may be only A, it may be only B, or it may be a combination of A and B. Further, in the present specification, in a case where three or more matters are connected and expressed by “and/or”, the same concept as “A and/or B” is applied.

All documents, patent applications, and technical standards described in the present specification are incorporated in the present specification by reference to the same extent in a case where it is specifically and individually described that the individual documents, the patent applications, and the technical standards are incorporated by reference.

Further, the following Supplementary Notes will be disclosed with respect to the above embodiments.

(Supplementary Note 1)

A medical service support device includes: a processor; and a memory that is connected to or built into the processor, in which the processor is configured to: derive, based on standard operation time, during which a medical device that is used in a medical device handling service is being operated, and on standby time for a medical device procedure service included in the medical device handling service, an operation rate reference value of the medical device; and output medical service support information obtained in accordance with a difference degree between an operation rate of the medical device and the operation rate reference value.

According to the configuration described in Supplementary Note 1, it is possible to support the efficient performance of the medical device handling service.

(Supplementary Note 2)

In the medical service support device according to Supplementary Note 1, the medical device is at least one of a medical management device used in the medical device procedure service, a medical accessory device that is attachably and detachably connected to the medical management device, or a washer that washes the medical accessory device.

What is claimed is:

1. A medical service support device comprising:
a processor; and
a memory that is connected to or built into the processor,
wherein the processor is configured to:
derive, based on standard operation time, during which a medical device that is used in an endoscope handling service is being operated, and on standby time for an endoscope procedure service included in the endoscope handling service, an operation rate reference value of the medical device; and
output medical service support information obtained in accordance with a difference degree between an operation rate of the medical device and the operation rate reference value.

2. The medical service support device according to claim 1,
wherein the medical service support information is information that includes medical device related information related to the medical device of which the operation rate is within an operation rate range defined based on the operation rate reference value.

3. The medical service support device according to claim 2,
wherein the medical device related information is information that includes information related to the medical device of which the operation rate is within the operation rate range during a time period when an operation degree of the medical device is equal to or higher than a first reference degree.

4. The medical service support device according to claim 3,
wherein the time period when the operation degree of the medical device is equal to or higher than the first reference degree is a time period when a value based on the number of endoscope procedure services is equal to or greater than a default value.

5. The medical service support device according to claim 3,
wherein the time period when the operation degree of the medical device is equal to or higher than the first reference degree is a time period when the number of times the standby time falls below a threshold value is equal to or greater than a reference number of times.

6. The medical service support device according to claim 2,
wherein the medical device related information is information that includes medical device quantity specification information capable of specifying a quantity of the medical devices of which the operation rate is within the operation rate range.

7. The medical service support device according to claim 1,
wherein the standard operation time is time based on operation time of the medical device during a time period when an operation degree of the medical device is equal to or higher than a second reference degree.

8. The medical service support device according to claim 1,
wherein the operation rate reference value is a value based on a ratio of the standard operation time with respect to time including shortest standby time, which is defined as shortest time for the standby time, and the standard operation time.

9. The medical service support device according to claim 8,
wherein the operation rate reference value is a value obtained by multiplying the ratio by an adjustment factor.

10. The medical service support device according to claim 1,
wherein the medical device is an information processing apparatus used together with an endoscope in the endoscope procedure service, and
operation time of the information processing apparatus is time from when a first endoscope procedure service, in which the information processing apparatus is used, is started to when the first endoscope procedure service is ended.

11. The medical service support device according to claim 1,
wherein the medical device is an endoscope, and
operation time of the endoscope is time from when a second endoscope procedure service, in which the endoscope is used, is started to when washing of the endoscope, which is used in the second endoscope procedure service, by a washer is ended.

12. The medical service support device according to claim 1,
wherein the medical device is a washer that washes an endoscope, and
operation time of the washer is time from when washing of the endoscope by the washer is started to when the washing of the endoscope by the washer is ended.

13. The medical service support device according to claim 1,
wherein a plurality of the medical devices are present, and
the standard operation time is time based on a statistical value obtained by using operation time of each of the plurality of medical devices.

14. The medical service support device according to claim 13,
wherein the medical device is provided at each of a plurality of medical sites where the endoscope handling service is performed, and
the statistical value is a value derived by using the operation time of each of the medical devices used in the plurality of medical sites.

15. The medical service support device according to claim 1,
wherein the medical service support information is information that includes notification information for notifying that an operation status of the medical device is tight.

16. The medical service support device according to claim 15,
wherein the notification information is information that includes a time slot, in which the operation status of the medical device is tight, and medical device quantity specification information capable of specifying a quantity of the medical devices operating during the time slot, and
the time slot and the medical device quantity specification information are presented by a presentation device in a comparable state.

17. The medical service support device according to claim 1,
wherein the processor is configured to:
derive the operation rate reference value for each type of endoscope procedure service; and
output the medical service support information for each type of endoscope procedure service.

18. The medical service support device according to claim 1,
wherein the medical service support information is information capable of being represented via at least one of a comment, a diagram, or a table by a presentation device.

19. A medical service support method comprising:
deriving, based on standard operation time, during which a medical device that is used in an endoscope handling service is being operated, and on standby time for an endoscope procedure service included in the endoscope handling service, an operation rate reference value of the medical device; and
outputting medical service support information obtained in accordance with a difference degree between an operation rate of the medical device and the operation rate reference value.

20. A non-transitory computer-readable storage medium storing a program executable by a computer to perform a process comprising:
deriving, based on standard operation time, during which a medical device that is used in an endoscope handling service is being operated, and on standby time for an endoscope procedure service included in the endoscope handling service, an operation rate reference value of the medical device; and
outputting medical service support information obtained in accordance with a difference degree between an operation rate of the medical device and the operation rate reference value.
